(19) United States
(12) Patent Application Publication
(10) Pub. No.: US 2011/0224148 A1
(43) Pub. Date: Sep. 15, 2011

(54) CARBOHYDRATE-POLYAMINO ACID-DRUG CONJUGATES

(75) Inventors: Mohammad Ahmadian, Carlsbad, CA (US); Wendy Taylor, San Diego, CA (US); Sang Van, San Diego, CA (US); Lei Yu, Carlsbad, CA (US)

(73) Assignee: NITTO DENKO CORPORATION, Osaka (JP)

(21) Appl. No.: 13/044,402

(22) Filed: Mar. 9, 2011

Related U.S. Application Data
(60) Provisional application No. 61/312,981, filed on Mar. 11, 2010.

(51) Int. Cl. A61K 47/48; A61P 35/00

(52) U.S. Cl. 514/19.3; 514/20.9

(57) ABSTRACT

Compositions that include a polymer conjugate and glucosamine operatively associated with the polymer conjugate, wherein the polymer conjugate includes a first drug, are described herein. Also disclosed herein are methods of using such compositions to treat, ameliorate, or diagnose a disease or condition such as cancer.

### RELATED APPLICATION INFORMATION

This application claims priority to U.S. Provisional Application Ser. No. 61/312,981, filed on Mar. 11, 2010, which is hereby incorporated herein by reference in its entirety for all purposes.

### BACKGROUND

1. Field

Disclosed herein are compositions and methods related to the field of organic chemistry, pharmaceutical chemistry, biochemistry, molecular biology and medicine. More specifically, embodiments described herein relate to compositions and methods for delivering a drug into a cell.

2. Description

Paclitaxel (PTX), extracted from the bark of the Pacific Yew tree, is an FDA-approved drug for the treatment of ovarian cancer and breast cancer. Wani et al., “Plant antitumor agents. VI. The isolation and structure of taxol, a novel antileukemic and antitumor agent from Taxus brevifolia,” J Am Chem. Soc. 1971, 93, 2325-7. However, it is believed that paclitaxel suffers from poor bioavailability. While multiple approaches to improve bioavailability have been attempted, many existing formulations are not entirely satisfactory.

### SUMMARY

Some embodiments herein are directed to a composition that can include a polymer conjugate and glucosamine that is operatively associated with the polymer conjugate. The polymer conjugate can include at least one recurring unit selected from Formula (I) and Formula (II):

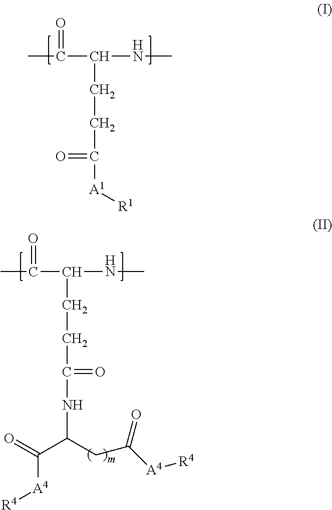

In Formula (I) and Formula (II), A¹ and A⁴ can each independently be oxygen or NR⁷, wherein R⁷ can be hydrogen or C₁₋₄ alkyl; R¹ can be a group that includes a first drug; each R⁴ can independently be hydrogen, a group that includes a first drug, a group that includes glucosamine, ammonium, or an alkali metal, with the proviso that one R⁴ is a group that includes a first drug and one R⁴ (in the same recurring unit of Formula (II)) is a hydrogen, a group that includes a first drug, a group that includes glucosamine, ammonium, or an alkali metal; and m can be 1 or 2. In some embodiments, m can be 2.

In some embodiments, the polymer conjugate can include at least one recurring unit that includes a group that includes glucosamine. In some embodiments, the at least one recurring unit can have a structure selected from Formula (III) and Formula (IV):

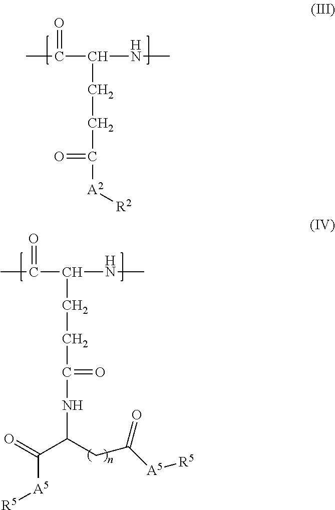

In Formula (III) and Formula (IV), each A² and each A⁵ can independently be oxygen or NR⁷, wherein R⁷ is hydrogen or C₁₋₄ alkyl; R² can be a group that includes glucosamine; each R⁵ can independently be hydrogen, a group that includes glucosamine, ammonium, or an alkali metal, with the proviso that at least one R⁵ is a group that includes glucosamine; and n can be 1 or 2.

In some embodiments, the polymer conjugate can also include at least one recurring unit having a structure selected from Formula (V) and Formula (VI):

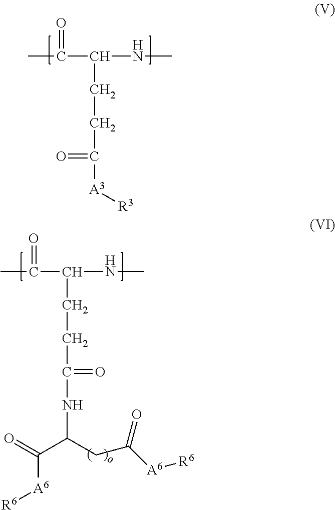

In Formula (V) and Formula (VI), A³ and A⁶ can each independently be oxygen or NR⁷, wherein R⁷ is hydrogen or C₁₋₄ alkyl; R³ and R⁶ can each independently be selected from a hydrogen, a C₁₋₁₀ alkyl group, a C₆₋₂₀ aryl group, an ammonium group, an alkali metal, a polydentate ligand, a polydentate ligand precursor with protected oxygen atoms, a group that comprises a targeting agent, a group that comprises an optical imaging agent, a group that comprises a magnetic resonance imaging agent, and a group that comprises a stabilizing agent; and o can be 1 or 2.

Other embodiments are directed to a method of making a composition that can include a polymer conjugate and glucosamine operatively associated with the polymer conjugate. The polymer conjugate can include at least one recurring unit selected from Formulae (I) and (II). These embodiments can include dissolving or partially dissolving a polymeric reactant including at least one recurring unit selected from Formulae (VII) and (VIII) in a solvent to form a dissolved or partially dissolved polymeric reactant.

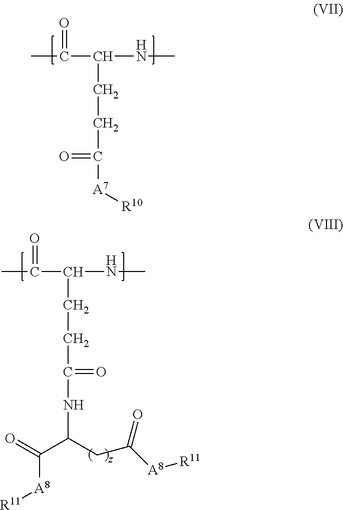

In Formulae (VII) and (VIII), each z can independently be 1 or 2; A⁷ and each A⁸ can be oxygen; and R¹⁰ and each R¹¹ can independently be selected from hydrogen, ammonium, and an alkali metal, for example lithium (Li), sodium (Na), potassium (K), rubidium (Rb), and cesium (Cs).

These embodiments can further include reacting the dissolved or partially dissolved polymeric reactant with a second reactant, wherein the second reactant can include the first drug; and intermixing the dissolved or partially dissolved polymeric reactant with a third reactant, wherein the third reactant can include glucosamine.

In some embodiments, the method of making the composition can further include reacting the dissolved or partially dissolved polymeric reactant with a fourth reactant, wherein the fourth reactant comprises at least one selected from a polydentate ligand, a polydentate ligand precursor with protected oxygen atoms, a group that comprises a third drug, a group that comprises a targeting agent, a group that comprises an optical imaging agent, a group that comprises a magnetic resonance imaging agent, and a group that comprises a stabilizing agent. In some embodiments, the fourth reactant may further include a substituent. The substituent may be selected from a hydroxy and an amine.

Yet other embodiments are directed to a method of treating, ameliorating, or diagnosing a disease or condition that can include administering an effective amount of a composition described herein to a mammal in need thereof.

These and other embodiments are described in greater detail below.

### BRIEF DESCRIPTION OF THE DRAWINGS

FIG. 1 schematically illustrates the synthesis of a composition that includes glucosamine, poly-(γ-L-glutamylglutamine) (PGGA), and paclitaxel.

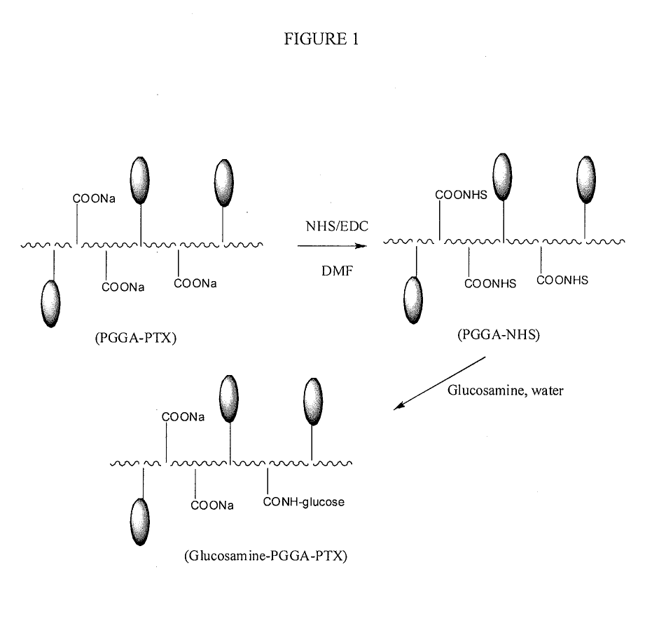

FIG. 2 illustrates a reaction scheme for the preparation of a composition that includes glucosamine, poly-(γ-L-glutamylglutamine) (PGGA), and paclitaxel.

FIG. 3 depicts a bar graph illustrating the results of an activated partial thromboplastin time (APTT) test utilizing several compositions described herein.

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety, unless stated otherwise. In the event that there are a plurality of definitions for a term, those in this section prevail unless stated otherwise.

The term “operatively associated” refers to an electronic interaction between a polymer conjugate, and glucosamine, as described herein. Such interaction may take the form of a chemical bond, including, but not limited to, a covalent bond, a polar covalent bond, an ionic bond, an electrostatic association, a coordinate covalent bond, an aromatic bond, a hydrogen bond, a dipole, or a van der Waals interaction. Those of ordinary skill in the art understand that the relative strengths of such interactions may vary widely. In some embodiments, a polymer conjugate is operatively associated with glucosamine when the polymer conjugate includes a recurring unit that includes a group that comprises glucosamine.

The term “polymer conjugate” is used herein in its ordinary sense and thus includes polymers that are attached to one or more types of biologically active agents or drugs, such as paclitaxel. For example, PGGA-PTX as described herein is a polymer conjugate in which poly-(γ-L-glutamylglutamine) (PGGA) is attached to paclitaxel (PTX). The polymer (e.g., PGGA) may be attached directly to the biologically active agent or drug (e.g., PTX), or may be attached through a linker group. The linker group may be a relatively small chemical moiety such as an ester or amide bond, or may be a larger chemical moiety, e.g., an alkyl ester linkage or an alkylene oxide linkage.

The terms “sugar” and “carbohydrate” as used herein each refer to monosaccharides, oligosaccharides and polysaccharides. A “polysaccharide” is a polymer comprised of recurring monosaccharide units joined by glycosidic bonds. An “oligosaccharide” is a polysaccharide comprised of 2-30 monosaccharide units joined by glycosidic bonds. A sugar can be naturally occurring or synthetic. Examples of sugars and/or carbohydrates include, but are not limited to, glucose (dextrose), fructose, galactose, xylose, ribose, sucrose, cellulose, cyclodextrin and starch.

The term “glucosamine” as used herein refers to an amino sugar having the following structure:

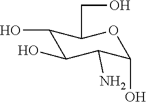

The term “glucosamine” also encompasses glucosamine that is covalently bonded to a polymer conjugate, either directly or through a linker group. Those skilled in the art will understand that when glucosamine is covalently bonded, the covalently bonded form of glucosamine has a structure that is slightly different from the structure shown above. The structure may differ in that one or more hydrogen atoms, one or more hydroxy groups and/or the —NH₂ group in the structure shown above are not present in the glucosamine, due to the covalent bond. A non-limiting example is shown below.

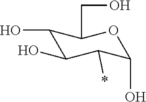

Those skilled in the art will appreciate that the asterisk in the above example indicates a point of attachment to the polymer conjugate or to a linker group that is attached to the polymer conjugate.

A “paramagnetic metal chelate” is a complex wherein a ligand is bound to a paramagnetic metal ion. Examples include, but are not limited to, 1,4,7,10-Tetraazacyclododecane-1,4,7,10-tetraacetic acid (DOTA)-Gd(III), DOTA-Yttrium-88, DOTA-Indium-111, diethylenetriaminepentaacetic acid (DTPA)-Gd(III), DTPA-yttrium-88, DTPA-Indium-111.

A “polydentate ligand” is a ligand that can bind itself through two or more points of attachment to a metal ion through, for example, coordinate covalent bonds. Examples of polydentate ligands include, but are not limited to, diethylenetriaminepentaacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox).

A “polydentate ligand precursor with protected oxygen atoms” is a polydentate ligand comprising oxygen atoms, such as the single-bonded oxygen atoms of carboxyl groups, that are protected with suitable protecting groups. Suitable protecting groups include, but are not limited to, lower alkyls, benzyls, and silyl groups.

A “stabilizing agent” is a substituent that enhances bioavailability and/or prolongs the half-life of a carrier-drug conjugate in vivo by rendering it more resistant to hydrolytic enzymes and less immunogenic. An exemplary stabilizing agent is polyethylene glycol (PEG).

As used herein, “Cₘ to Cₙ,” in which “m” and “n” are integers that refer to the number of carbon atoms in a group or the number of carbons in a ring(s). That is, the group or ring can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—, CH₃CH(CH₃)CH₂— and (CH₃)₃C—. If no “m” and “n” are designated, the broadest range described in the definitions provided herein is to be assumed.

As used herein, “alkyl” refers to a straight or branched fully saturated (no double or triple bonds) hydrocarbon group, for example, a group having the general formula —CₙH₂ₙ₊₁. The alkyl group may have 1 to 50 carbon atoms (whenever it appears herein, a numerical range such as “1 to 50” refers to each integer in the given range; e.g., “1 to 50 carbon atoms” means that the alkyl group may consist of 1 carbon atom, 2 carbon atoms, 3 carbon atoms, etc., up to and including 50 carbon atoms, although the present definition also covers the occurrence of the term “alkyl” where no numerical range is designated). The alkyl group may also be a medium size alkyl having 1 to 30 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “aryl” refers to a hydrocarbon monocyclic or multicyclic aromatic ring system that has a fully delocalized pi-electron system throughout all the rings. Examples of aryl groups include, but are not limited to, benzene, naphthalene and azulene. The ring(s) of the aryl group may have 5 to 50 carbon atoms. The aryl group may be substituted or unsubstituted.

As used herein, “heteroaryl” refers to a monocyclic or multicyclic aromatic ring system (a ring system with fully delocalized pi-electron system throughout all the rings) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain 5 to 50 atoms in the ring(s), 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. A heteroaryl group may be substituted or unsubstituted. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine.

Unless otherwise indicated, when a substituent is “optionally substituted,” or “substituted” it is meant that the substituent is a group that may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxy, alkoxy, aryloxy, acyl, ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. The protecting groups that may form the protective derivatives of the above substituents are known to those of skill in the art and may be found in references such as Greene and Wuts, Protective Groups in Organic Synthesis, 3^{rd} Ed., John Wiley & Sons, New York, N.Y., 1999, which is hereby incorporated by reference in its entirety.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound having one or more double bond(s) generating geometrical isomers that can be defined as E or Z each double bond may independently be E or Z a mixture thereof. Likewise, all tautomeric forms are also intended to be included.

As used herein, the abbreviations for any protective groups, amino acids and other compounds are, unless indicated otherwise, in accord with their common usage, recognized abbreviations, or the IUPAC-IUP Commission on Biochemical Nomenclature (See, Biochem. 11:942-944 (1972)).

Administered hydrophobic anticancer drugs, therapeutic proteins and polypeptides often suffer from poor bio-availability. In some cases it has been theorized that such poor bio-availability may be due to incompatibility of bi-phasic solutions of hydrophobic drugs and aqueous solutions and/or rapid removal of these molecules from blood circulation by enzymatic degradation. A variety of systems have been used for the delivery of biomolecules, imaging agents, and therapeutic agents, such as hydrophobic anticancer drugs. For example, such systems include capsules, liposomes, microparticles, nanoparticles, and polymers.

Approaches to improving the bioavailability of paclitaxel include formulating the paclitaxel in a mixture of Cremophor-EL and dehydrated ethanol (1:1, v/v) and creating an emulsification using high-shear homogenization. Sparreboom et al., “Cremophor EL-mediated Alteration of Paclitaxel Distribution in Human Blood: Clinical Pharmacokinetic Implications,” Cancer Research 1999, 59, 1454-1457 and Constantinides et al. “Formulation Development and Antitumor Activity of a Filter-Sterilizable Emulsion of Paclitaxel.” Pharmaceutical Research 2000, 17, 175-182. Commericially available formulations include Taxol™ (Bristol-Myers Squibb) and Abraxane® (American Pharmaceutical Partners, Inc.). In some cases, Taxol may result in inadequate delivery of effective drug levels and high toxicity. Additionally, although Taxol™ has demonstrated clinical efficacy in non-small-cell lung cancer (NSCLC), it can cause severe side effects including acute hypersensitivity reactions and peripheral neuropathies.

A variety of polyester-based biodegradable systems have also been characterized and studied. Polylactic acid (PLA), polyglycolic acid, and their copolymer polylactic-co-glycolic acid (PLGA) are some of the most well-characterized biomaterials with regard to design and performance for drug-delivery applications. See Uhrich, K. E., et al., “Polymeric Systems for Controlled Drug Release,” Chem. Rev. 1999, 99, 3181-3198; Panyam J, et al., “Biodegradable nanoparticles for drug and gene delivery to cells and tissue,” Adv Drug Deliv Rev. 2003, 55, 329-47. Also, 2-hydroxypropyl methacrylate (HPMA) has been used to create a polymer for drug-delivery applications. Biodegradable systems based on polyorthoesters have also been investigated. See Heller, J., et al., “Poly(ortho esters): synthesis, characterization, properties and uses,” Adv. Drug Del. Rev. 2002, 54, 1015-1039. Polyanhydride systems have also been investigated. Such polyanhydrides are typically biocompatible and may degrade in vivo into relatively non-toxic compounds that are eliminated from the body as metabolites. See Kumar, N., et al., “Polyanhydrides: an overview.” Adv. Drug Del. Rev. 2002, 54, 889-91. Polymer-paclitaxel conjugates have been advanced in several clinical trials (Ruth Duncan “The Dawning era of polymer therapeutics.” Nature Reviews Drug Discovery 2003, 2, 347-360).

One technique that has been studied for increasing the efficacy of administered proteins and other small molecule agents entails conjugating the administered agent with a polymer, such as a polyethylene glycol (“PEG”) molecule, that can provide protection from enzymatic degradation in vivo. Such “PEGylation” often improves the circulation time and, hence, bio-availability of an administered agent. PEG has shortcomings in certain respects, however. For example, because PEG is a linear polymer, the steric protection afforded by PEG can be limited, as compared to branched polymers. Another shortcoming of PEG is that it is generally amenable to derivatization at its two terminals. This limits the number of other functional molecules (e.g. those helpful for protein or drug delivery to specific tissues) that can be readily operatively associated with PEG.

Amino acid-based polymers have also been considered as a potential source of new biomaterials. Poly-amino acids having good biocompatibility have been investigated to deliver low molecular-weight compounds. A relatively small number of polyglutamic acids and copolymers have been identified as candidate materials for drug delivery. See Bourke, S. L., et al., “Polymers derived from the amino acid L-tyrosine: polycarbonates, polyarylates and copolymers with poly(ethylene glycol).” Adv. Drug Del. Rev., 2003, 55, 447-466.

Polyglutamic acid (PGA) is a polymer that can be used for solubilizing hydrophobic anticancer drugs. Many anti-cancer drugs conjugated to PGA have been reported. See Chun Li, “Poly(L-glutamic acid)-anticancer drug conjugates,” Adv. Drug Del. Rev., 2002, 54, 695-713. However, none are currently FDA-approved. Accordingly, there is a long-felt need for improved anticancer drug formulations and methods of delivering them.

Some embodiments herein are directed to a composition that can include a polymer conjugate and glucosamine that is operatively associated with the polymer conjugate. In some embodiments, the polymer conjugate can include at least one recurring unit having a structure selected from Formula (I) and Formula (II):

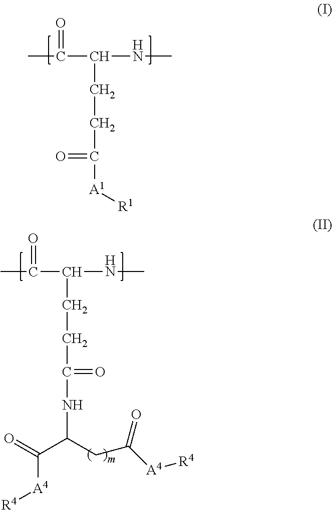

In Formula (I) and Formula (II), A¹ and A⁴ can each independently be oxygen or NR⁷, wherein R⁷ can be hydrogen or C₁₋₄ alkyl; R¹ can be a group that includes a first drug; each R⁴ can independently be hydrogen a group that includes a first drug, a group that includes glucosamine, ammonium, or an alkali metal, with the proviso that one R⁴ is a group that includes a first drug and one R⁴ (in the same recurring unit of Formula (II)) is a hydrogen, a group that comprises a first drug, a group that includes glucosamine, ammonium, or an alkali metal; and m can be 1 or 2. In some embodiments, m can be 2. In some embodiments, the alkali metal can be lithium (Li), sodium (Na), potassium (K), rubidium (Rb), or cesium (Cs). In some embodiments, the alkali metal can be sodium.

In some embodiments, the composition can include at least one recurring unit of Formula (I). In other embodiments, the composition can include at least one recurring unit of Formula (II). In some embodiments, the recurring unit of Formula (II) can have a structure represented by one of Formulae (IIa)-(IIc):

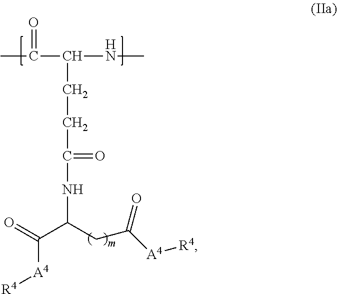

wherein one R⁴ is a group that includes a first drug and one R⁴ is H, ammonium, or an alkali metal.

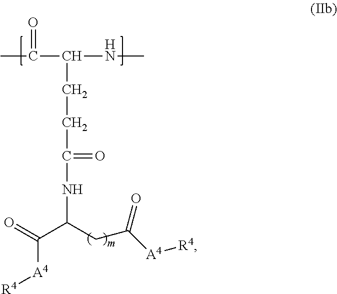

wherein one R⁴ is a group that includes a first drug and one R⁴ is a group that includes glucosamine.

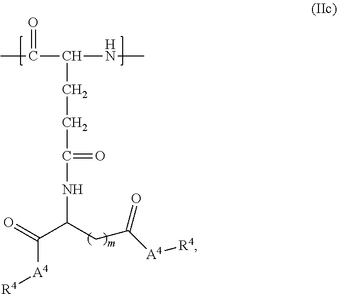

wherein both R⁴ is a group that includes a first drug.

A variety of drugs may be used for the first drug. In some embodiments, the first drug can be a first hydrophobic drug. In some embodiments, the first hydrophobic drug can include an anticancer drug. In some embodiments, the anticancer drug can be selected from a taxane, a camptotheca, and an anthracycline. Examples of taxanes include, but are not limited to, paclitaxel and docetaxel. In some embodiments, the taxane can be paclitaxel. In some embodiments where the first drug includes paclitaxel, the paclitaxel can attach to the recurring unit of Formula (I) and/or Formula (II) at the oxygen atom attached to the C2′-carbon of the paclitaxel. In other embodiments, the paclitaxel can attach to the recurring unit of Formula (I) and/or Formula (II) at the oxygen atom attached to the C7-carbon of the paclitaxel. In some embodiments, the camptotheca can be camptothecin. In some embodiments, the anthracycline can be doxorubicin. In some embodiments, the composition can include multiple first drugs. When the composition includes multiple first drugs, each first drug can be the same or different. For example, when a polymer conjugate described herein contains more than one recurring unit of Formula (I) and/or Formula (II), the first drug of one recurring unit can be the same as the first drug of a second recurring unit. As an example, a polymer conjugate can have one recurring unit of Formula (I) with a first drug that includes paclitaxel and another recurring unit of Formula (II) with a first drug that includes paclitaxel. Likewise, when a polymer conjugate described herein contains more than one recurring unit of Formula (I) and/or Formula (II), the first drug of one recurring unit can be different from the first drug of a second recurring unit. For example, a polymer conjugate can have a recurring unit of Formula (I) with a first drug that includes paclitaxel and another recurring unit of Formula (I) with a first drug that includes camptothecin.

The amount of the first drug present in the composition can vary over a wide range. In some embodiments, the composition can include a total amount of the first drug in the range of about 10 mole % to about 70 mole % based on the total moles of recurring units in the composition. In other embodiments, the composition can include a total amount of the first drug in the range of about 30 mole % to about 60 mole % based on the total moles of recurring units in the composition. In still other embodiments, the composition can include a total amount of the first drug in the range of about 20 mole % to about 50 mole % based on the total moles of recurring units in the composition.

In some embodiments, the composition can include a total amount of first drug in the range of about 1% to about 50% (weight/weight), about 1% to about 40% (weight/weight), about 1% to about 30% (weight/weight) about 1% to about 20% (weight/weight) or 1% to about 10% (weight/weight) based on the mass ratio of the first drug to the composition (the weight of the first drug is accounted for in the composition).

The composition can also include glucosamine that is operatively associated with the polymer conjugate. The glucosamine can be operatively associated with the polymer conjugate in various ways. For example, the glucosamine can be operatively associated with the polymer conjugate via an electrostatic association, via a direct bond, or via a linker group. In some embodiments, the glucosamine can be electrostatically associated with the polymer conjugate.

Some embodiments herein are directed to a composition that can include a polymer conjugate that includes a recurring unit of Formula (I) and glucosamine that is mixed in and/or electrostatically associated with the polymer conjugate. Other embodiments herein are directed to a composition that can include a polymer conjugate that includes a recurring unit of Formula (II) and glucosamine that is electrostatically associated with the polymer conjugate. Some embodiments herein are directed to a composition that can include a polymer conjugate that includes a recurring unit of Formula (IIa) and glucosamine that is electrostatically associated with the polymer conjugate. Other embodiments herein are directed to a composition that can include a polymer conjugate that includes a recurring unit of Formula (IIb) and glucosamine that is electrostatically associated with the polymer conjugate. Still other embodiments are directed to a composition that can include a polymer conjugate that includes a recurring unit of Formula (IIc) and glucosamine that is electrostatically associated with the polymer conjugate. In some embodiments, where glucosamine is electrostatically associated with the polymer conjugate, the glucosamine can be a compound that includes glucosamine in a covalently bonded form. In some embodiments where glucosamine is electrostatically associated with the polymer conjugate, the glucosamine can have the following structure:

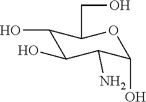

In other embodiments, the polymer conjugate can include at least one recurring unit that includes a group that comprises glucosamine. Some embodiments are directed to a composition that can include a polymer conjugate operatively associated with glucosamine, wherein the polymer conjugate includes a recurring unit of Formula (I) and a recurring unit of Formula (IIb), wherein one R⁴ is a group that includes a first drug and one R⁴ is a group that includes glucosamine. Some embodiments are directed to a composition that can include a polymer conjugate operatively associated with glucosamine, wherein the polymer conjugate includes a recurring unit of Formula (IIb), wherein one R⁴ is a group that includes a first drug and one R⁴ is a group that includes glucosamine.

In some embodiments, the recurring unit that includes a group that comprises glucosamine can have a structure selected from Formula (III) and Formula (IV):

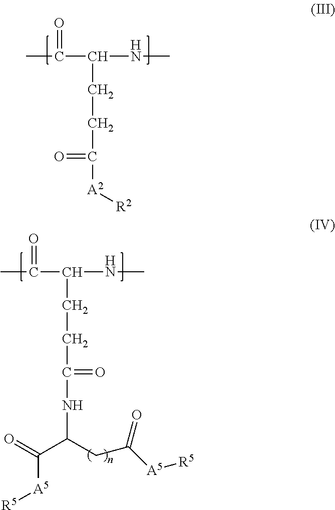

In Formula (III) and Formula (IV), each A² and each A⁵ can independently be oxygen or NR⁷, wherein R⁷ is hydrogen or C₁₋₄ alkyl; R² can be a group that includes glucosamine; each R⁵ can independently be hydrogen, a group that includes glucosamine, ammonium, or an alkali metal, with the proviso that at least one R⁵ is a group that includes glucosamine; and n can be 1 or 2. In some embodiments, n can be 2. In some embodiments, the alkali metal can be lithium (Li), sodium (Na), potassium (K), rubidium (Rb), or cesium (Cs). In some embodiments, the alkali metal can be sodium. Those of ordinary skill in the art will appreciate that when glucosamine forms a part of a recurring unit of Formula (III) or Formula (IV), the glucosamine may have a structure that is slightly modified as described herein.

Some embodiments herein are directed to a composition that can include a polymer conjugate operatively associated with glucosamine, wherein the polymer conjugate includes a recurring unit of Formula (I) and a recurring unit of Formula (III). Other embodiments herein are directed to a composition that can include a polymer conjugate operatively associated with glucosamine, wherein the polymer conjugate includes a recurring unit of Formula (I) and a recurring unit of Formula (IV). Yet other embodiments herein are directed to a composition that can include a polymer conjugate operatively associated with glucosamine, wherein the polymer conjugate includes a recurring unit of Formula (I) and one or more recurring units of Formulae (IIb), (III), and/or (IV).

In some embodiments, the polymer conjugate can include a recurring unit of Formula (IIa), wherein one R⁴ is a group that includes a first drug and one R⁴ is H, ammonium, or an alkali metal. Some embodiments herein are directed to a polymer conjugate operatively associated with glucosamine, wherein the polymer conjugate can include a recurring unit of Formula (IIa) and a recurring unit of Formula (IIb), wherein one R⁴ is a group that includes a first drug and one R⁴ is a group that includes glucosamine. Other embodiments herein are directed to a polymer conjugate operatively associated with glucosamine, wherein the polymer conjugate can include a recurring unit of Formula (IIa) and a recurring unit of Formula (III). Yet other embodiments are directed to a polymer conjugate operatively associated with glucosamine, wherein the polymer conjugate can include a recurring unit of Formula (IIa) and a recurring unit of Formula (IV). Yet still other embodiments are directed to a polymer conjugate operatively associated with glucosamine, wherein the polymer conjugate can include a recurring unit of Formula (IIa) and one or more recurring units of Formulae (IIb), (III), and/or (IV).

In some embodiments, the polymer conjugate can include a recurring unit of Formula (IIc), wherein both R⁴ is a group that includes a first drug. Some embodiments herein are directed to a polymer conjugate operatively associated with glucosamine, wherein the polymer conjugate can include a recurring unit of Formula (IIc) and a recurring unit of Formula (IIb), wherein one R⁴ is a group that includes a first drug and one R⁴ is a group that includes glucosamine. Other embodiments herein are directed to a polymer conjugate operatively associated with glucosamine, wherein the polymer conjugate can include a recurring unit of Formula (IIc) and a recurring unit of Formula (III). Yet other embodiments are directed to a polymer conjugate operatively associated with glucosamine, wherein the polymer conjugate can include a recurring unit of Formula (IIc) and a recurring unit of Formula (IV). Yet still other embodiments are directed to a polymer conjugate operatively associated with glucosamine, wherein the polymer conjugate can include a recurring unit of Formula (IIc) and one or more recurring units of Formulae (IIb), (III), and/or (IV).

In other embodiments, the polymer conjugate can include a recurring unit of Formula (IIb), wherein one R⁴ is a group that includes a first drug and one R⁴ is a group that includes glucosamine. Some embodiments herein are directed to a polymer conjugate operatively associated with glucosamine, wherein the polymer conjugate can include a recurring unit of Formula (IIb). Other embodiments herein are directed to a polymer conjugate operatively associated with glucosamine, wherein the polymer conjugate can include a recurring unit of Formula (IIb) and a recurring unit of Formula (III). Yet other embodiments are directed to a polymer conjugate operatively associated with glucosamine, wherein the polymer conjugate can include a recurring unit of Formula (IIb) and a recurring unit of Formula (IV). Yet still other embodiments are directed to a polymer conjugate operatively associated with glucosamine, wherein the polymer conjugate can include a recurring unit of Formula (IIb) and one or more recurring units of Formulae (III), and/or (IV).

In some embodiments, the polymer conjugate can include at least one recurring unit of Formula (I) and at least one recurring unit of Formula (III). In some embodiments, the polymer conjugate can include at least one recurring unit of Formula (II) and at least one recurring unit of Formula (IV). In some embodiments, the polymer conjugate can include at least one recurring unit of Formula (II), wherein one R⁴ is hydrogen, ammonium, an alkali metal, or a group that includes a first drug and the other R⁴ is a group that includes a first drug, and at least one recurring unit of Formula (IV). In other embodiments, the polymer conjugate can include at least one recurring unit of Formula (II), wherein one R⁴ is a group that includes a first drug and the other R⁴ is a group that includes glucosamine. In some embodiments, the polymer conjugate can include at least one recurring unit of Formula (II) and at least one recurring unit of Formula (III). In some embodiments, the polymer conjugate can include at least one recurring unit of Formula (I) and at least one recurring unit of Formula (IV). In still other embodiments, the polymer conjugate can include at least one recurring unit of Formula (I) and at least one recurring unit of Formula (II), wherein at least one R⁴ is a group that can include glucosamine.

In some embodiments, a polymer conjugate can include a recurring unit of Formula (IV), wherein one R⁵ is a group that includes glucosamine and one R⁵ is an alkali metal. In other embodiments, a polymer conjugate can include a recurring unit of Formula (IV), wherein one R⁵ is a group that includes glucosamine and one R⁵ is hydrogen. In yet other embodiments, a polymer conjugate can include a recurring unit of Formula (IV), wherein both R⁵ are groups that include glucosamine.

An example of a recurring unit of Formula (IV) is shown below as Formula (IVa):

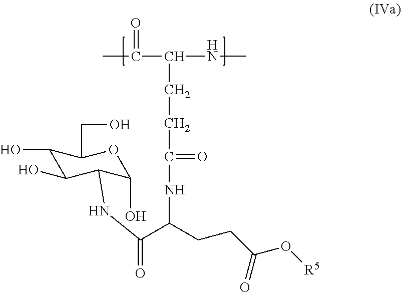

In some embodiments, the polymer conjugate can include a recurring unit of Formula (IVa), wherein R⁵ is a group that includes glucosamine and a recurring unit of Formula (II) wherein one R⁴ is a group that includes first drug and one R⁴ is a group that comprises glucosamine.

The amount of glucosamine present in the composition can vary over a wide range. In some embodiments, the composition can include a total amount of glucosamine in the range of about 1 mole % to about 90 mole % based on the total moles of recurring units in the composition. In other embodiments, the composition can include a total amount of glucosamine in the range of about 50 mole % to about 80 mole % based on the total moles of recurring units in the composition. In still other embodiments, the composition can include a total amount of glucosamine in the range of about 10 mole % to about 70 mole % based on the total moles of recurring units in the composition.

In some embodiments, the composition can include a total amount of glucosamine in the range of about 1% to about 50% (weight/weight) based on the mass ratio of the glucosamine to the composition (the weight of the glucosamine are accounted for in the composition). In other embodiments, the composition can include a total amount of glucosamine in the range of about 1% to about 40% (weight/weight) based on the mass ratio of the glucosamine to the composition. In still other embodiments, the composition can include a total amount of glucosamine in the range of about 1% to about 30% (weight/weight) based on the mass ratio of the glucosamine to the composition. In yet still other embodiments, the composition can include a total amount of glucosamine in the range of about 1% to about 20% (weight/weight) based on the mass ratio of the glucosamine to the composition. In some embodiments, the composition can include a total amount of glucosamine in the range of about 1% to about 10% (weight/weight) based on the mass ratio of the glucosamine to the composition.

Those of ordinary skill in the art will appreciate that paclitaxel (PTX) operatively associated with poly-(γ-L-glutamylglutamine) (PGGA) has been shown to increase activated partial thromboplastin time (APTT), a measure of the length of time it takes for blood to clot. While the APTT for an untreated sample of human plasma has been measured to be about 40 seconds, the APTT of human plasma treated with PGGA-PTX has been measured to be about 350 seconds. Indeed, petechiae (broken capillary blood vessels, indicative of, e.g., clotting factor deficiencies) have been observed in nude mice when PGGA-paclitaxel formulations are injected at 350 mg/kg. This effect is considered to be heparin-like. Heparin, a highly-sulfated glycosaminoglycan, is widely used as an injectable anticoagulant, and is believed to have the highest negative charge density of any known biological molecule. Although this application is not bound by theory, it is believed that this “heparin-like” effect of PGGA-PTX on APTT is due to the highly negative charge of PGGA-PTX. Indeed, measurements show that PGGA-PTX may have a surface charge of approximately −20 mV. It has been reported that polycations are generally cytotoxic, haemolytic and can activate complement, whereas polyanions are less cytotoxic, but can cause anticoagulant activity and can also stimulate cytokine release. See Duncan, R., “The Dawning of Polymer Therapeutics,” Nat. Rev. Drug Discov. Vol. 2 pp. 347-360 (May 2003).

It has been advantageously and unexpectedly discovered that a composition that includes a polymer conjugate and glucosamine operatively associated with the polymer conjugate, as described herein, can include a total amount of glucosamine that is effective to yield a composition that exhibits a reduced APTT as compared to an otherwise comparable composition that lacks glucosamine. For example, a composition that includes an anionic polymer conjugate and glucosamine operatively associated with the polymer conjugate can include a total amount of glucosamine that is effective to yield an APTT in the range of from about 50 seconds to about 60 seconds. Those skilled in the art will appreciate that reference to an APTT of a composition herein will be understood as referring to an APTT of a sample of human plasma that has been treated or intermixed with the composition.

Thus, some embodiments are directed to a composition that includes a polymer conjugate and glucosamine operatively associated with the polymer conjugate (for example, a polymer conjugate described herein that includes at least one recurring unit selected from Formula (I) and Formula (II)), wherein the glucosamine is present in the composition in a total amount that is effective to yield a composition having an APTT that is less than the APTT of an otherwise comparable composition that lacks glucosamine. In some embodiments, the glucosamine is present in the composition in a total amount that is effective to yield a composition having an APTT that is no more than about 20% of the APTT of an otherwise comparable composition that lacks glucosamine. For example, where the APTT of an otherwise comparable composition that lacks glucosamine is about 350 seconds, the APTT of a composition that includes a polymer conjugate and glucosamine operatively associated with the polymer conjugate (for example, a polymer conjugate described herein that includes at least one recurring unit selected from Formula (I) and Formula (II)) can include a total amount of glucosamine that is effective to yield a composition having an APTT that is no more than about 20% of about 350 seconds. Thus, in some embodiments, the APTT of a composition that includes a polymer conjugate and glucosamine operatively associated with the polymer conjugate (for example, a polymer conjugate described herein that includes at least one recurring unit selected from Formula (I) and Formula (II)) can include a total amount of glucosamine that is effective to yield a composition having an APTT that is no more than about 70 seconds. In some embodiments, the composition that includes a polymer conjugate and glucosamine operatively associated with the polymer conjugate can include a total amount of glucosamine that is effective to yield a composition having an APTT that is in the range of from about 14% to about 17% of the APTT of an otherwise comparable composition that lacks glucosamine. Those skilled in the art will understand that a “comparable” composition is a control material in which the polymer conjugate has approximately the same average number and type of recurring units (except any glucosamine covalently bonded to the same recurring units is replaced with the needed number of hydrogen atoms to fill the valency of the atom to which the glucosamine is attached) as that of the subject polymer conjugate (comprising a recurring unit of the Formula (I) and a recurring unit of the Formula (II)) to which it is being compared.

In other embodiments, the glucosamine is present in the composition in a total amount that is effective to yield a composition having an APTT that is at least about 80% less than the APTT of an otherwise comparable composition that lacks glucosamine. For example, where the APTT of an otherwise comparable composition that lacks glucosamine is about 350 seconds, the APTT of a composition that includes a polymer conjugate and glucosamine operatively associated with the polymer conjugate (for example, a polymer conjugate described herein that includes at least one recurring unit selected from Formula (I) and Formula (II)) can include a total amount of glucosamine that is effective to yield a composition having an APTT that is at least about 80% less than 350 seconds. Thus, in some embodiments, the APTT of a composition that includes a polymer conjugate and glucosamine operatively associated with the polymer conjugate (for example, a polymer conjugate described herein that includes at least one recurring unit selected from Formula (I) and Formula (II)) can include a total amount of glucosamine that is effective to yield a composition having an APTT that is at least about 280 seconds less than about 350 seconds, i.e., at most about 70 seconds. In some embodiments, the glucosamine is present in the composition in a total amount that is effective to yield a composition having an APTT that is in the range of from about 80% to about 85% less than the APTT of an otherwise comparable composition that lack glucosamine.

In yet other embodiments, a composition that includes a polymer conjugate and glucosamine operatively associated with the polymer conjugate, as described herein, has a total amount of glucosamine that is effective to yield a composition having an APTT that is an APTT that is no more than 50% greater than the APTT of an untreated sample of human plasma. For example, where the APTT of an untreated sample of human plasma is about 40 seconds, the APTT of a composition that includes a polymer conjugate and glucosamine that is operatively associated with the polymer conjugate (for example, a polymer conjugate described herein that includes at least one recurring unit selected from Formula (I) and Formula (II)) can include a total amount of glucosamine that is effective to yield a composition having an APTT that is no more than 50% greater than about 40 seconds. Thus, in some embodiments, the APTT of a composition that includes a polymer conjugate and glucosamine operatively associated with the polymer conjugate (for example, a polymer conjugate described herein that includes at least one recurring unit selected from Formula (I) and Formula (II)) can include a total amount of glucosamine that is effective to yield a composition having an APTT that is no more than 50% greater than about 40 seconds, i.e., no more than about 60 seconds. In some embodiments, a composition that includes a polymer conjugate and glucosamine operatively associated with the polymer conjugate can include a total amount of glucosamine that is effective to yield a composition having an APTT that is in the range of from about 25% greater to about 50% greater than the APTT of an untreated sample of normal human plasma.

In any of these embodiments, the APTT of the composition can be measured using commercially-available coagulation tests, such as the STart® 4 Coagulation Analyzer (Diagnostica Stago). For example, the APTT can be measured on a mixture of 120 μL of normal human plasma intermixed with 30 μL of the composition that has been dissolved in saline to a concentration of at least 5 mg/mL.

Those skilled in the art may appreciate that some embodiments may be directed to a composition that includes a different blood procoagulant instead of glucosamine. This blood procoagulant may be operatively associated with a polymer conjugate in the same manner described herein with respect to glucosamine. Examples of suitable blood procoagulants include, but are not limited to, thrombin, fibrin, fibrinogen, hemostatic agents, desmopressin, and coagulation factors.

The polymer conjugate can also include at least one recurring unit having a structure selected from Formula (V) and Formula (VI):

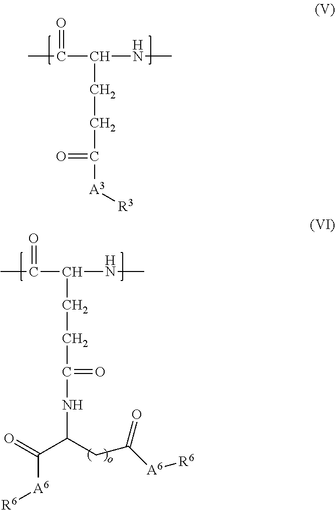

In Formula (V) and Formula (VI), A³ and A⁶ can each independently be oxygen or NR⁷, wherein R⁷ is hydrogen or C₁₋₄ alkyl; R³ and R⁶ can each independently be selected from a hydrogen, a C₁₋₁₀ alkyl group, a C₆₋₂₀ aryl group, an ammonium group, an alkali metal, a polydentate ligand, a polydentate ligand precursor with protected oxygen atoms, a group that comprises a targeting agent, a group that comprises an optical imaging agent, a group that comprises a magnetic resonance imaging agent, and a group that comprises a stabilizing agent; and o can be 1 or 2.

In some embodiments, the compositions and/or polymer conjugates described herein can include an alkali metal. In some embodiments, each of R³ and R⁶ can be independently selected to comprise an alkali metal, such as lithium (Li), sodium (Na), potassium (K), rubidium (Rb), and cesium (Cs). In some embodiments, the alkali metal can be sodium. In some embodiments, each of R³ and R⁶ can comprise hydrogen, a C₁₋₁₀ alkyl group, a C₆₋₂₀ aryl group or an ammonium group.

When A³ is oxygen and R³ is hydrogen, then the recurring unit of Formula (V) is a recurring unit of glutamic acid. When o is 1, each A⁶ is oxygen, and each R⁶ is hydrogen, the recurring unit of Formula (VI) is a recurring unit of L-aspartyl-glutamine. In some embodiments, o is 2. When o is 2, each A⁶ is oxygen, and each R⁶ is hydrogen or an alkali metal, the recurring unit of Formula (VI) is a recurring unit of L-glutamyl-glutamine, as shown below.

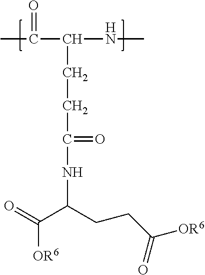

The composition can include any combination of recurring units of Formulae (I), (II), (III), (IV), (V), and/or (VI) as described herein. In some embodiments, the composition can include at least one recurring unit of Formula (I), at least one recurring unit of Formula (III), and at least one recurring unit of Formula (V). In other embodiments, the composition can include at least one recurring unit of Formula (II), at least one recurring unit of Formula (IV), and at least one recurring unit of Formula (VI). In some embodiments, the recurring unit of Formula (IV) has the structure of Formula (IVa).

In some embodiments, at least one R³ can independently be a group that can include an agent. In some embodiments, at least one R⁶ can independently be a group that can include an agent. Many types of agents can be used. For example, the agent(s) may be selected from a targeting agent, an optical imaging agent, a magnetic resonance imaging agent, and a stabilizing agent.

In some embodiments, the agent can include an optical imaging agent. Examples of optical imaging agent include, but are not limited to, an acridine dye, a coumarine dye, a rhodamine dye, a xanthene dye, a cyanine dye, and a pyrene dye. A non-limiting list of specific optical imaging agents includes Texas Red, Alexa Fluor® dye, BODIPY® dye, Fluorescein, Oregon Green® dye, and Rhodamine Green™ dye, which are commercially available or readily prepared by methods known to those skilled in the art.

In some embodiments, the agent can comprise a targeting agent. In some embodiments, the targeting agent can be one or more selected from an arginine-glycine-aspartate (RGD) peptide, fibronectin, folate, galactose, an apolipoprotein, insulin, transferrin, a fibroblast growth factor (FGF), an epidermal growth factor (EGF), and an antibody. In some embodiments, the targeting agent can interact with a receptor selected from αᵥ,β₃-integrin, folate, asialoglycoprotein, a low-density lipoprotein (LDL), an insulin receptor, a transferrin receptor, a fibroblast growth factor (FGF) receptor, an epidermal growth factor (EGF) receptor, and an antibody receptor. In some embodiments, the arginine-glycine-aspartate (RGD) peptide can be cyclic (fKRGD).

In some embodiments, the agent can comprise a magnetic resonance imaging agent. In some embodiments, the magnetic resonance imaging agent can include a paramagnetic metal compound. For example, the magnetic resonance imaging agent may include a Gd(III) compound. In some embodiments, the Gd(III) compound can be selected from:

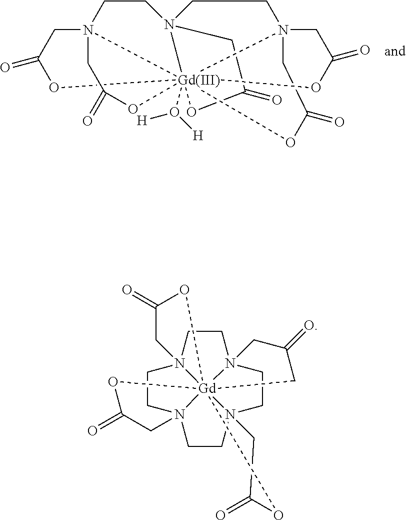

In some embodiments, the agent can comprise a stabilizing agent. An example of a suitable stabilizing agent is polyethylene glycol.

In some embodiments, the polymer conjugate can comprise a polydentate ligand. In some embodiments, each of R³ and R⁶ can be independently selected to comprise a group that includes a polydentate ligand. In some embodiments, the polydentate ligand may be capable of reaction with a paramagnetic metal to form a magnetic resonance imaging agent. The polydentate ligand may comprise several carboxylic acid and/or carboxylate groups. In some embodiments, the polydentate ligand can be selected from:

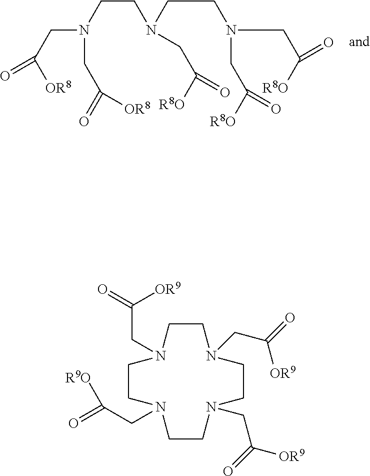

wherein each R⁸ and each R⁹ can be independently selected from hydrogen, ammonium, and an alkali metal.

In some embodiments, the polymer conjugate comprises a polydentate ligand precursor. In some embodiments, each of R³ and R⁶ can be independently selected to comprise a group that includes a polydentate ligand precursor. In such embodiments, the oxygen atoms of the polydentate ligand may be protected by a suitable protecting group. Suitable protecting groups include, but are not limited to, lower alkyls, benzyls, and silyl groups. One example of a polydentate ligand precursor having protecting groups is provided as follows:

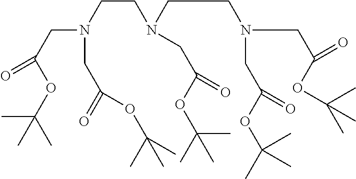

The amount of agent(s) (e.g., a targeting agent, an optical imaging agent, a magnetic resonance imaging agent, and/or a stabilizing agent) present in the composition can vary over a wide range. Additionally, the amount of a ligand or a ligand precursor present in the composition can vary over a wide range. In some embodiments, the composition comprises an amount of a targeting agent, an optical imaging agent, a magnetic resonance imaging agent, a stabilizing agent, a ligand, and/or a ligand precursor in the range of about 0.1% to about 50% (weight/weight) based on the mass ratio of the agent(s), ligand, and/or ligand precursor to the composition (the weight of the agent(s), ligand, and/or ligand precursor is accounted for in the composition). In other embodiments, the composition comprises an amount of an agent(s), a ligand, and/or a ligand precursor in the range of about 1% to about 40% (weight/weight) based on the mass ratio of the agent(s), ligand, and/or ligand precursor to the composition. In still other embodiments, the composition comprises an amount of an agent(s), a ligand, and/or a ligand precursor in the range of about 1% to about 30% (weight/weight) based on the mass ratio of the agent(s), ligand, and/or ligand precursor to the composition. In yet still other embodiments, the composition comprises an amount of an agent(s), a ligand, and/or a ligand precursor in the range of about 1% to about 20% (weight/weight) based on the mass ratio of the agent(s), ligand, and/or ligand precursor to the composition. In some embodiments, the composition comprises an amount of an agent(s), a ligand, and/or a ligand precursor in the range of about 1% to about 10% (weight/weight) based on the mass ratio of the agent(s), ligand, and/or ligand precursor to the composition. In other embodiments, the composition comprises an amount of an agent(s), a ligand, and/or a ligand precursor in the range of about 5% to about 40% (weight/weight) based on the mass ratio of the agent(s), ligand, and/or ligand precursor to the composition. In still other embodiments, the composition comprises an amount of an agent(s), a ligand, and/or a ligand precursor in the range of about 10% to about 30% (weight/weight) based on the mass ratio of the agent(s), ligand, and/or ligand precursor to the composition. In yet still other embodiments, the composition comprises an amount of an agent(s), a ligand, and/or a ligand precursor in the range of about 20% to about 40% (weight/weight) based on the mass ratio of the agent(s), ligand, and/or ligand precursor to the composition. In some embodiments, the composition comprises an amount of an agent(s), a ligand, and/or a ligand precursor in the range of about 30% to about 50% (weight/weight) based on the mass ratio of the agent(s), ligand, and/or ligand precursor to the composition.

As described herein, glucosamine may be operatively associated with the polymer conjugate in a variety of different ways. In some embodiments, the glucosamine is operatively associated with the polymer conjugate through an electrostatic association. The glucosamine may be operatively associated with the polymer conjugate at various positions relative to the polymer conjugate. Such positions may be fixed (e.g., at the middle, ends, or side chains of the polymer conjugate) or relative, e.g., the polymer conjugate may exhibit a configuration in a particular medium (such as an aqueous medium) such that is has interior and exterior portions. In some embodiments, glucosamine may be operatively associated with a side chain moiety of the polymer conjugate. In other embodiments, the glucosamine may be operatively associated with an end or terminal recurring unit of the polymer conjugate. In yet other embodiments, glucosamine may be operatively associated with the middle of the polymer conjugate. In still yet other embodiments, glucosamine may be operatively associated with the backbone of the polymer conjugate. In some embodiments, glucosamine may be operatively associated with an exterior moiety or surface of the polymer conjugate. In some embodiments, glucosamine may be operatively associated with an interior moiety or surface of the polymer conjugate. In some embodiments, glucosamine can be at least partially contained within the polymer conjugate. In other embodiments, glucosamine may be substantially completely contained within the polymer conjugate.

A group that comprises a first drug, a group that comprises glucosamine, a group that comprises a targeting agent, a group that comprises an optical imaging agent, a group that comprises a magnetic resonance imaging agent, a group that comprises a polydentate ligand, a group that comprises a polydentate ligand precursor, and/or a group that comprises a stabilizing agent may be chemically bonded to the polymer conjugate in many different ways. In some embodiments, the aforementioned compounds can be directly attached to the polymer conjugate, e.g., to a recurring unit of Formulae (I), (II), (III), (IV), (V), and/or (VI), respectively. In some embodiments, one or more of a group that comprises a first drug, a group that comprises glucosamine, a group that comprises a targeting agent, a group that comprises an optical imaging agent, a group that comprises a magnetic resonance imaging agent, a group that comprises a polydentate ligand, a group that comprises a polydentate ligand precursor, and a group that comprises a stabilizing agent can be directly attached to the polymer conjugate through an oxygen, a sulfur, a nitrogen and/or carbon atom of the agent, drug, or group. In some embodiments, the glucosamine can be conjugated to polymer conjugate through its nitrogen atom.

In other embodiments, one or more of a group that comprises a first drug, a group that comprises glucosamine, a group that comprises a targeting agent, a group that comprises an optical imaging agent, a group that comprises a magnetic resonance imaging agent, a group that comprises a polydentate ligand, a group that comprises a polydentate ligand precursor, and a group that comprises a stabilizing agent can further include a linker group. In some embodiments, the group that comprises the first drug further can include a linker group. In other embodiments, the group that comprises glucosamine can further include a linker group. In some embodiments, the group that comprises a targeting agent, the group that comprises an optical imaging agent, the group that comprises a magnetic resonance imaging agent, the group that comprises a polydentate ligand, the group that comprises a polydentate ligand precursor, and/or the group that comprises a stabilizing agent can further include a linker group. A linker group is a group that attaches, for example, the agent (or the compound that comprises the agent) to the polymer conjugate. In some embodiments, one or more of the aforementioned compounds can be attached to the polymer conjugate, e.g., to a recurring unit of Formulae (I), (II), (III) (IV), (V), and/or (VI), respectively, through a linker group. The linker group may be relatively small. For instance, the linker group may comprise an amine, an amide, an ether, an ester, a hydroxyl group, a carbonyl group, or a thiol ether group. Alternatively, the linker group may be relatively large. For instance, the linker group may comprise an alkyl group, an ether group, an aryl group, an aryl(C₁₋₆ alkyl) group (e.g., phenyl-(CH₂)₁₋₄-), a heteroaryl group, or a heteroaryl(C₁₋₆ alkyl) group. In some embodiments, the linker can be —NH(CH₂)₁₋₄—NH—. In other embodiments, the linker can be —(CH₂)₁₋₄-aryl-NH—. The linker group can be attached to one or more of a group that comprises a drug, a group that comprises glucosamine, a group that comprises a targeting agent, a group that comprises an optical imaging agent, a group that comprises a magnetic resonance imaging agent, a group that comprises a polydentate ligand, a group that comprises a polydentate ligand precursor, or a group that comprises a stabilizing agent at any suitable position. For example, the linker group can be attached in place of a hydrogen at a carbon of one of the aforementioned compounds. The linker group can be added to the compounds using methods known to those skilled in the art.

Compositions comprising a recurring unit of Formulae (I), (II), (III), (IV), (V), and/or (VI) as described herein can be copolymers comprising two or more different recurring units of the Formulae (I), (II), (III), (IV), (V), and/or (VI). Further, compositions comprising a recurring unit of the Formulae (I), (II), (III), (IV), (V), and/or (VI) can be copolymers that comprise other recurring units that are not of the Formulae (I), (II), (III), (IV), (V), and/or (VI). A broad variety of other recurring units may be included in the compositions described herein. The number of recurring units of the Formulae (I), (II), (III), (IV), (V), and/or (VI) in the compositions can vary over a broad range, such as in the range of from about 50 to about 5,000, or in the range of from about 100 to about 2,000.

The percentage of recurring units of Formula (I) in the composition, based on the total number of recurring units, may vary over a wide range. In some embodiments, the composition may comprise a percentage of recurring units of Formula (I) of up to about 99 mole %, based on the total moles of recurring units in the composition. In other embodiments, the composition may comprise a percentage of recurring units of Formula (I) in the range of from about 1 mole % to about 99 mole %, based on the total moles of recurring units in the composition. In still other embodiments, the composition may comprise a percentage of recurring units of Formula (I) in the range of from about 1 mole % to about 50 mole % based on the total moles of recurring units in the composition. In yet still other embodiments, the composition may comprise a percentage of recurring units of Formula (I) in the range of from about 1 mole % to about 30 mole % based on the total moles of recurring units in the composition. In some embodiments, the composition may comprise a percentage of recurring units of Formula (I) in the range of from about 1 mole % to about 20 mole % based on the total moles of recurring units in the composition. In other embodiments, the composition may comprise a percentage of recurring units of Formula (I) in the range of from about 1 mole % to about 10 mole % based on the total moles of recurring units in the composition.

The percentage of recurring units of Formula (II) in the composition, based on the total number of recurring units, may vary over a wide range. In some embodiments, the composition may comprise a percentage of recurring units of Formula (II) of up to about 99 mole %, based on the total moles of recurring units in the composition. In other embodiments, the composition may comprise a percentage of recurring units of Formula (II) in the range of from about 1 mole % to about 99 mole %, based on the total moles of recurring units in the composition. In still other embodiments, the composition may comprise a percentage of recurring units of Formula (II) in the range of from about 1 mole % to about 50 mole % based on the total moles of recurring units in the composition. In yet still other embodiments, the composition may comprise a percentage of recurring units of Formula (II) in the range of from about 1 mole % to about 30 mole % based on the total moles of recurring units in the composition. In some embodiments, the composition may comprise a percentage of recurring units of Formula (II) in the range of from about 1 mole % to about 20 mole % based on the total moles of recurring units in the composition. In other embodiments, the composition may comprise a percentage of recurring units of Formula (II) in the range of from about 1 mole % to about 10 mole % based on the total moles of recurring units in the composition.

The percentage of recurring units of Formula (III) in the composition, based on the total number of recurring units, may vary over a wide range. In some embodiments, the composition may comprise a percentage of recurring units of Formula (III) of up to about 99 mole %, based on the total moles of recurring units in the composition. In other embodiments, the composition may comprise a percentage of recurring units of Formula (III) in the range of from about 1 mole % to about 99 mole %, based on the total moles of recurring units in the composition. In still other embodiments, the composition may comprise a percentage of recurring units of Formula (III) in the range of from about 1 mole % to about 50 mole % based on the total moles of recurring units in the composition. In yet still other embodiments, the composition may comprise a percentage of recurring units of Formula (III) in the range of from about 1 mole % to about 30 mole % based on the total moles of recurring units in the composition. In some embodiments, the composition may comprise a percentage of recurring units of Formula (III) in the range of from about 1 mole % to about 20 mole % based on the total moles of recurring units in the composition. In other embodiments, the composition may comprise a percentage of recurring units of Formula (III) in the range of from about 1 mole % to about 10 mole % based on the total moles of recurring units in the composition.

The percentage of recurring units of Formula (IV) in the composition, based on the total number of recurring units, may vary over a wide range. In some embodiments, the composition may comprise a percentage of recurring units of Formula (IV) of up to about 99 mole %, based on the total moles of recurring units in the composition. In other embodiments, the composition may comprise a percentage of recurring units of Formula (IV) in the range of from about 1 mole % to about 99 mole %, based on the total moles of recurring units in the composition. In still other embodiments, the composition may comprise a percentage of recurring units of Formula (IV) in the range of from about 1 mole % to about 50 mole % based on the total moles of recurring units in the composition. In yet still other embodiments, the composition may comprise a percentage of recurring units of Formula (IV) in the range of from about 1 mole % to about 30 mole % based on the total moles of recurring units in the composition. In some embodiments, the composition may comprise a percentage of recurring units of Formula (IV) in the range of from about 1 mole % to about 20 mole % based on the total moles of recurring units in the composition. In other embodiments, the composition may comprise a percentage of recurring units of Formula (IV) in the range of from about 1 mole % to about 10 mole % based on the total moles of recurring units in the composition.

The percentage of recurring units of Formula (V) in the composition, based on the total number of recurring units, may vary over a wide range. In some embodiments, the composition may comprise a percentage of recurring units of Formula (V) of up to about 99 mole %, based on the total moles of recurring units in the composition. In other embodiments, the composition may comprise a percentage of recurring units of Formula (V) in the range of from about 1 mole % to about 99 mole %, based on the total moles of recurring units in the composition. In still other embodiments, the composition may comprise a percentage of recurring units of Formula (V) in the range of from about 1 mole % to about 50 mole % based on the total moles of recurring units in the composition. In yet still other embodiments, the composition may comprise a percentage of recurring units of Formula (V) in the range of from about 1 mole % to about 30 mole % based on the total moles of recurring units in the composition. In some embodiments, the composition may comprise a percentage of recurring units of Formula (V) in the range of from about 1 mole % to about 20 mole % based on the total moles of recurring units in the composition. In other embodiments, the composition may comprise a percentage of recurring units of Formula (V) in the range of from about 1 mole % to about 10 mole % based on the total moles of recurring units in the composition.

The percentage of recurring units of Formula (VI) in the composition, based on the total number of recurring units, may vary over a wide range. In some embodiments, the composition may comprise a percentage of recurring units of Formula (VI) of up to about 99 mole %, based on the total moles of recurring units in the composition. In other embodiments, the composition may comprise a percentage of recurring units of Formula (VI) in the range of from about 1 mole % to about 99 mole %, based on the total moles of recurring units in the composition. In still other embodiments, the composition may comprise a percentage of recurring units of Formula (VI) in the range of from about 1 mole % to about 50 mole % based on the total moles of recurring units in the composition. In yet still other embodiments, the composition may comprise a percentage of recurring units of Formula (VI) in the range of from about 1 mole % to about 30 mole % based on the total moles of recurring units in the composition. In some embodiments, the composition may comprise a percentage of recurring units of Formula (VI) in the range of from about 1 mole % to about 20 mole % based on the total moles of recurring units in the composition. In other embodiments, the composition may comprise a percentage of recurring units of Formula (VI) in the range of from about 1 mole % to about 10 mole % based on the total moles of recurring units in the composition.

In some embodiments, the composition can include two or more recurring units selected from a recurring unit of the Formula (I), a recurring unit of the Formula (II), a recurring unit of the Formula (III), a recurring unit of the Formula (IV), a recurring unit of the Formula (V), and a recurring unit of the Formula (VI). In other embodiments, the composition can include three or more recurring units selected from a recurring unit of the Formula (I), a recurring unit of the Formula (II), a recurring unit of the Formula (III), a recurring unit of the Formula (IV), a recurring unit of the Formula (V), and a recurring unit of the Formula (VI). In still other embodiments, the composition can include four or more recurring units selected from a recurring unit of the Formula (I), a recurring unit of the Formula (II), a recurring unit of the Formula (III), a recurring unit of the Formula (IV), a recurring unit of the Formula (V), and a recurring unit of the Formula (VI). In yet still other embodiments, the composition can include five or more recurring units selected from a recurring unit of the Formula (I), a recurring unit of the Formula (II), a recurring unit of the Formula (III), a recurring unit of the Formula (IV), a recurring unit of the Formula (V), and a recurring unit of the Formula (VI). In some embodiments, the composition can include six different recurring units of the Formulae (I), (II), (III), (IV), (V), and (VI).

The amount of each recurring unit (e.g., mole percent) present in the composition can vary greatly, as set forth above. In some embodiments, selection of an amount of any one recurring unit of the Formulae (I), (II), (III), (IV), (V), and/or (VI) can be independent of the selection of an amount of a different recurring unit of the Formulae (I), (II), (III), (IV), (V), and/or (VI).

In some embodiments, the amounts of the agent(s), the amount of glucosamine, the amount of first drug, and the percentage of the recurring unit of the Formulae (I), (II), (III), (IV), (V), and/or (VI) in the composition can be selected to provide a solubility of the composition that is greater than that of a comparable polyglutamic acid conjugate that comprises substantially the same amount of the agent(s), the amount of glucosamine, and/or drugs. The range of pH values over which the composition, comprising recurring units of the Formulae (I), (II), (III), (IV), (V), and/or (VI), has greater solubility than that of a comparable polyglutamic acid conjugate may be narrow or broad. Solubility is measured by forming a composition solution comprising at least 5 mg/mL of the composition in 0.9 wt. % aqueous NaCl at about 22° C., and determining the optical clarity. In some embodiments, the composition is soluble over a pH range of at least about three pH units. In other embodiments, the composition is soluble over a pH range of at least about 8 pH units. In yet other embodiments, the composition is soluble over a pH range of at least about 9 pH units. In yet still other embodiments, the pH range over which the composition is soluble includes at least one pH value in the range of about 2 to about 5, e.g., at pH=2, pH=3, pH=4 and/or pH=5. Preferably, the pH range over which the composition is soluble is broader than the pH range over which the comparable polyglutamic acid conjugate is soluble. For example, in some embodiments, the composition is soluble over a pH range that is at least about one pH unit broader, preferably at least about two pH units broader, than the pH range over which the comparable polyglutamic acid conjugate is soluble.

The amount of composition placed in solution to measure solubility can also vary greatly. In some embodiments, solubility is measured when the tested composition solution comprises at least about 5 mg/mL of the composition. In other embodiments, solubility is measured when the tested composition solution comprises at least about 10 mg/mL of the composition. In still other embodiments, solubility is measured when the tested composition solution comprises at least about 25 mg/mL of the composition. In yet still other embodiments, solubility is measured when the tested composition solution comprises at least about 100 mg/mL of the composition. In some embodiments, solubility is measured when the tested composition solution comprises at least about 150 mg/mL of the composition. Those skilled in the art will understand that the comparable polyglutamic acid conjugate is tested at about the same concentration as that of the tested composition.

Some embodiments are directed to methods of making the compositions described herein. Some embodiments are directed to methods of making a composition that can include a polymer conjugate, wherein the polymer conjugate can include at least one recurring unit selected from Formulae (I) and (II), and wherein the polymer conjugate may be operatively associated with glucosamine. These embodiments can include dissolving or partially dissolving a polymeric reactant including at least one recurring unit selected from Formulae (VII) and (VIII) in a solvent to form a dissolved or partially dissolved polymeric reactant.

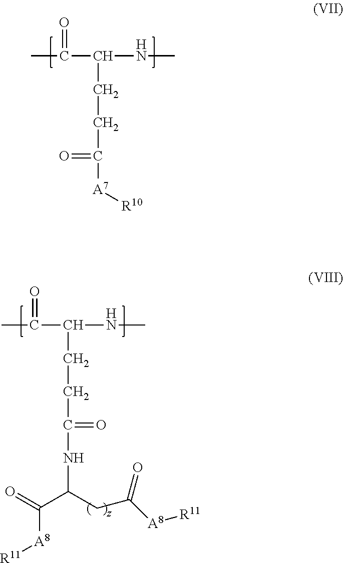

In Formulae (VII) and (VIII), each z can independently be 1 or 2; A⁷ and each A⁸ can be oxygen; and R¹⁰ and each R¹¹ can independently be selected from hydrogen, ammonium, and an alkali metal, for example lithium (Li), sodium (Na), potassium (K), rubidium (Rb), and cesium (Cs).

These embodiments can further include reacting the dissolved or partially dissolved polymeric reactant with a second reactant, wherein the second reactant can include the first drug; and intermixing the dissolved or partially dissolved polymeric reactant with a third reactant, wherein the third reactant can include glucosamine.

The second reactant may comprise many different types of drugs. In some embodiments, the first drug can be a first hydrophobic drug. In some embodiments, the first hydrophobic drug can include an anticancer drug. In some embodiments, the anticancer drug can be selected from a taxane, a camptotheca, and an anthracycline. In some embodiments, the taxane can be paclitaxel or docetaxel. In some embodiments, the taxane can be paclitaxel. In some embodiments where the first drug includes paclitaxel, the paclitaxel can attach to the recurring unit of Formula (I) and/or Formula (II) at the oxygen atom attached to the C2′-carbon of the paclitaxel. In other embodiments, the paclitaxel can attach to the recurring unit of Formula (I) and/or Formula (II) at the oxygen atom attached to the C7-carbon of the paclitaxel. In some embodiments, the camptotheca can be camptothecin. In other embodiments, the anthracycline can be doxorubicin.

In some embodiments, the second reactant can include a substituent selected from hydroxy and amine. In some embodiments, the third reactant can include a substituent selected from hydroxy and amine.

In some embodiments, the dissolved or partially dissolved polymeric reactant can be reacted with at least a portion of the second reactant before the dissolved or partially dissolved reactant is intermixed with at least a portion of the third reactant. In other embodiments, the dissolved or partially dissolved polymeric reactant can be reacted with at least a portion of the second reactant after the dissolved partially dissolved reactant is intermixed with at least a portion of the third reactant. In some embodiments, the dissolved or partially dissolved polymeric reactant can be reacted with at least a portion of the second reactant at about the same time as the dissolved or partially dissolved polymeric reactant is intermixed with at least a portion of the third reactant. In other embodiments, the third reactant can be added without isolating the intermediate compound that forms after the addition of the second reactant.

In some embodiments, a polymeric reactant comprising a recurring unit of the Formula (VII) can be produced starting with polyglutamic acid. Alternatively, in other embodiments, the polymeric reactant may be created by first converting the starting polyglutamic acid material into its salt form. The salt form of polyglutamic acid can be obtained by reacting polyglutamic acid with a suitable base, e.g., sodium bicarbonate. The weight average molecular weight of the polyglutamic acid is not limited, but is preferably from about 10,000 to about 500,000 Daltons, and more preferably from about 25,000 to about 300,000 Daltons.

In some embodiments, a polymeric reactant comprising a recurring unit of the Formula (VIII) can be produced starting with polyglutamic acid and an amino acid such as asparatic and/or glutamic acid. Alternatively, in other embodiments, the polymeric reactant may be created by first converting the starting polyglutamic acid material into its salt form. The salt form of polyglutamic acid can be obtained by reacting polyglutamic acid with a suitable base, e.g., sodium bicarbonate. An amino acid moiety can be attached to the pendant carboxylic acid group of the polyglumatic acid. The weight average molecular weight of the polyglutamic acid is not limited, but is preferably from about 10,000 to about 500,000 Daltons, and more preferably from about 25,000 to about 300,000 Daltons. Such a reaction may be used to create poly-(γ-L-aspartyl-glutamine) or poly-(γ-L-glutamyl-glutamine).

In some embodiments, the amino acid can be protected by a protecting group before attachment to the polyglutamic acid. One example of a protected amino acid moiety suitable for this reaction is L-aspartic acid di-t-butyl ester hydrochloride, shown below:

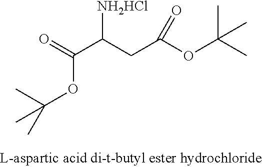

Reaction of the polyglutamic acid with the amino acid may take place in the presence of any suitable solvent. In some embodiments, the solvent can be an aprotic solvent. In some embodiments, the solvent is N,N′-dimethylformamide. In some embodiments, a coupling agent such as EDC, DCC, CDI, DSC, HATU, HBTU, HCTU, PyBOP®, PyBroP®, TBTU, and BOP can be used in the reaction between the polyglutamic acid and the amino acid. In other embodiments, polyglutamic acid and an amino acid can be reacted using a catalyst (e.g., DMAP).

The composition may be recovered and/or purified by methods known to those skilled in the art. For example, the solvent may be removed by suitable methods, for instance, rotary evaporation. Additionally, the reaction mixture may be filtered into an acidic water solution to induce precipitation. The resultant precipitate can then be filtered, and washed with water.

In some embodiments, a polymeric reactant comprising a recurring unit of the Formula (VII) can also include a recurring unit of Formula (VIII). One method for forming a polymeric reactant comprising a recurring unit of Formula (VII) and a recurring unit of Formula (VIII) is by starting with polyglutamic acid and reacting it with an amino acid such as asparatic and/or glutamic acid, in an amount that is less than 1.0 equivalents of the amino acid based on polyglutamic acid. For example, in some embodiments, 0.7 equivalents of an amino acid based on the polyglutamic acid can be reacted with polyglutamic acid, so that about 70% of the recurring units of the resulting polymer include the amino acid. As discussed above, the oxygen atoms of the amino acid can be protected using a suitable protecting group. In some embodiments, the amino acid may be L-aspartic acid or L-glutamic acid. In other embodiments, the oxygen atoms of the amino acid can be protected with t-butyl groups. If the oxygen atoms of the amino acid are protected, the protecting groups can be removed using known methods such as a suitable acid (e.g., trifluoroacetic acid).

In some embodiments, the method of making the composition can further include reacting the dissolved or partially dissolved polymeric reactant with a fourth reactant, wherein the fourth reactant comprises at least one selected from a polydentate ligand, a polydentate ligand precursor with protected oxygen atoms, a group that comprises a third drug, a group that comprises a targeting agent, a group that comprises an optical imaging agent, a group that comprises a magnetic resonance imaging agent, and a group that comprises a stabilizing agent. In some embodiments, the fourth reactant may further include a substituent. The substituent may be selected from a hydroxy and an amine.

In some embodiments, the fourth reactant can include an agent selected from a compound that comprises a polydentate ligand, a polydentate ligand precursor with protected oxygen atoms, a group that comprises a targeting agent, a group that comprises an optical imaging agent, a group that comprises a magnetic resonance imaging agent, and a group that comprises a stabilizing agent.

In some embodiments, the fourth reactant can include a group that comprises a targeting agent. In some embodiments, the targeting agent can be selected from an arginine-glycine-aspartate (RGD) peptide, fibronectin, folate, galactose, an apolipoprotein, insulin, transferrin, a fibroblast growth factor (FGF), an epidermal growth factor (EGF), and an antibody. In some embodiments, the targeting agent can interact with a receptor selected from αᵥ,β₃-integrin, folate, asialoglycoprotein, a low-density lipoprotein (LDL), an insulin receptor, a transferrin receptor, a fibroblast growth factor (FGF) receptor, an epidermal growth factor (EGF) receptor, and an antibody receptor. In some embodiments, the arginine-glycine-aspartate (RGD) peptide can be cyclic (fKRGD).

In some embodiments, the fourth reactant can include a group that comprises an optical imaging agent, including those described herein. In some embodiments, the optical imaging agent may be selected from an acridine dye, a coumarine dye, a rhodamine dye, a xanthene dye, a cyanine dye, and a pyrene dye.

In some embodiments, the fourth reactant can include a group that comprises a stabilizing agent. In some embodiments, the stabilizing agent can be polyethylene glycol.

In some embodiments, the fourth reactant can include a group that comprises a magnetic resonance imaging agent. In some embodiments, the magnetic resonance imaging agent can include a paramagnetic metal compound. Preferably, the compound that comprises the agent comprises a Gd(III) compound. Exemplary Gd(III) compounds include the following:

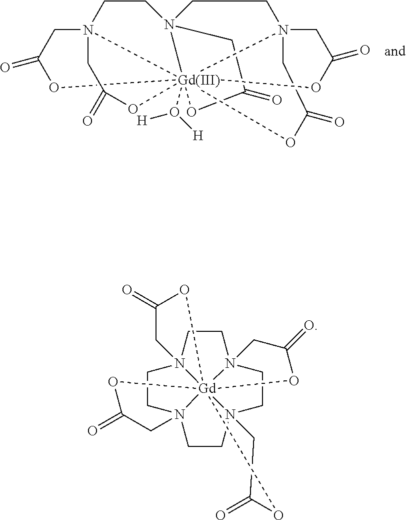

In some embodiments, the fourth reactant can include a polydentate ligand. Any suitable polydentate ligand may be used. In some embodiments, the polydentate ligand may be capable of reaction with a paramagnetic metal to form a magnetic resonance imaging agent. For example, the polydentate ligand may comprise several carboxylic acid and/or carboxylate groups. For example, polydentate ligands of the following structures may be operatively associated with the polymer:

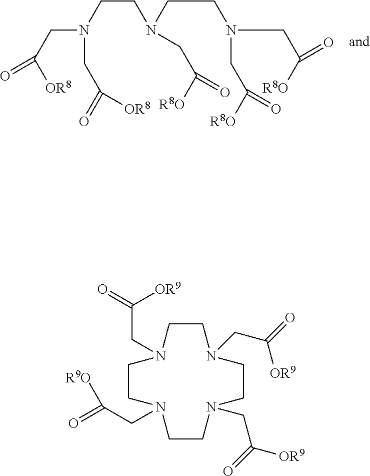

wherein each R⁸ and each R⁹ can be independently hydrogen, ammonium, or an alkali metal.

In some embodiments, the fourth reactant can include a polydentate ligand precursor. In other embodiments, a polydentate ligand precursor having protecting groups may be operatively associated with the polymer. Such a precursor has its oxygen atoms protected by a suitable protecting group(s). Suitable protecting groups include, but are not limited to, lower alkyls, benzyls, and silyl groups. One example of a polydentate ligand precursor having protecting groups is provided as follows:

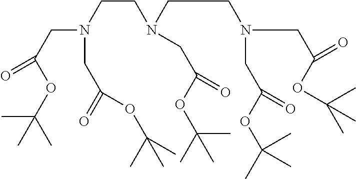

In some embodiments, the dissolved or partially dissolved polymeric reactant can be reacted with at least a portion of the second reactant and/or intermixed with at least a portion of the third reactant before reacting with at least a portion of a fourth reactant. In some embodiments, the dissolved or partially dissolved polymeric reactant is reacted with at least a portion of a fourth reactant before reacting before reacting with at least a portion of the second reactant and/or intermixing with at least a portion of the third reactant. In some embodiments, the dissolved or partially dissolved polymeric reactant is reacted with at least a portion of the fourth reactant at about the same time it is reacted with at least a portion of the second reactant and/or intermixed with at least a portion of the third reactant.

In some embodiments, a method of making the composition can include reacting and/or intermixing the dissolved or partially dissolved polymeric reactant with the second reactant and/or third reactant in the presence of a coupling agent. A coupling reagent may also be present for reaction with the fourth reactant. Any suitable coupling agent may be used. In some embodiments, the coupling agent can be selected from 1-ethyl-3-(3-dimethylaminopropyl)-carbodiimide (EDC), 1,3-dicyclohexyl carbodiimide (DCC), 1,1′-carbonyl-diimidazole (CDI), N,N′-disuccinimidyl carbonate (DSC), N-[(dimethylamino)-1H-1,2,3-triazolo-[4,5-b]pyridine-1-yl-methylene]-N-methylmethanaminium hexafluorophosphate N-oxide (HATU), 2-[(1H-benzotriazol-1-yl)-1,1,3,3-tetramethylaminium hexafluorophosphate (HBTU), 2-[(6-chloro-1H-benzotriazol-1-yl)-1,1,3,3-tetramethylaminium hexafluorophosphate (HCTU), benzotriazole-1-yl-oxy-tri s-pyrrolidino-phosphonium hexafluorophosphate (PyBOP®), bromo-tris-pyrrolidino-phosphonium hexafluorophosphate (PyBroP®), 2-[(1H-benzotriazol-1-yl)-1,1,3,3-tetramethylaminium tetrafluoroborate (TBTU), and benzotriazol-1-yl-oxy-tris-(dimethylamino)phosphonium hexafluorophosphate (BOP).

Any suitable solvent that allows the reaction to take place may be used. In some embodiments, the solvent may be a polar aprotic solvent. For instance, the solvent may be selected from N,N-dimethylformamide (DMF), dimethyl sulfoxide (DMSO), N-methyl-2-pyridone (NMP), and N,N-dimethylacetamide (DMAc).

In other embodiments, the reaction may further include reacting the dissolved or partially dissolved polymeric reactant in the presence of a catalyst. Any catalyst that promotes the reaction may be used. In some embodiments, the catalyst may comprise 4-dimethylaminopyridine (DMAP).

Operative association of a group that comprises a targeting agent, a group that comprises an optical imaging agent, a group that comprises a magnetic resonance imaging agent, a group that comprises a polydentate ligand, a group that comprises a polydentate ligand precursor and/or a group that comprises a stabilizing agent to the polymer acid or its salt form may be carried out in various ways, e.g., by covalently bonding the group comprising an agent, a polydentate ligand, and/or a polydentate ligand precursor with protected oxygen atoms to various polymers. One method for operatively associating the aforementioned groups to the polymer is by using heat (e.g., heat from using a microwave method). Alternatively, operative association may take place at room temperature. Appropriate solvents, coupling agents, catalysts, and/or buffers as generally known to those skilled in the art and/or as described herein may be used to form the composition. As with polyglutamic acid, both the salt or acid form of the polymer obtained from polyglutamic acid and/or salt and an amino acid can be used as starting material for forming the composition.

Suitable agents that can be operatively associated with the polymer conjugates described herein include but are not limited to drugs, optical agents, targeting agents, magnetic resonance imaging agents (e.g., paramagnetic metal compounds), stabilizing agents, polydentate ligands, and polydentate ligand precursors with protected oxygen atoms.

As an example, in some embodiments, the polymer conjugate can be operatively associated with an optical imaging agent such as those described herein. In some embodiments, the optical agent can be Texas Red-NH₂.

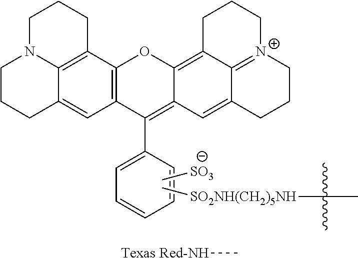

In one particular embodiment, a suitable polymeric reactant capable of forming a composition described herein (e.g., a polymer obtained from polyglutamic acid and/or salt and an amino acid) may be reacted with DCC, Texas Red-NH₂ dye, pyridine, and 4-dimethylaminopyridine. The mixture can be heated using a microwave method. In some embodiments, the reaction can be heated up to a temperature in the range of about 100° to about 150° C. In other embodiments, the time the materials are heated ranges from about 5 to about 40 minutes. If desired, the reaction mixture can be cooled to room temperature. Suitable methods known to those skilled in the art can be used to isolate and/or purify the composition. For instance, reaction mixture can be filtered into an acidic water solution. Any precipitate that forms can then be filtered and washed with water. Optionally, the precipitate can be purified by any suitable method. For example, the precipitate can be transferred into acetone and dissolved, and the resulting solution can be filtered again into a sodium bicarbonate solution. If desired, the resulting reaction solution can be dialyzed in water using a cellulose membrane and the composition can be lyophilized and isolated.

In some embodiments, a suitable polymeric reactant capable of forming the composition described herein can be operatively associated with a drug (e.g., an anticancer drug).

The drug can be operatively associated with the suitable polymeric reactant using the methods described above with respect to Texas-Red.

In some embodiments, paclitaxel, preferably in the presence of a coupling agent (e.g, EDC and/or DCC) and a catalyst (e.g, DMAP), can be reacted with a suitable polymeric reactant capable of forming a composition described herein in a solvent (e.g, an aprotic solvent such as DMF). Additional agents, such as pyridine or hydroxybenzotriazole may be used. In some embodiments, the reaction may take place over the period of 0.5-2 days. Suitable methods known to those skilled in the art can be used to isolate and/or purify the composition. For example, the reaction mixture can be poured into an acidic solution to form a precipitate. Any precipitate that forms can then be filtered and washed with water. Optionally, the precipitate can be purified by any suitable method. For example, the precipitate can be transferred into acetone and dissolved, and the resulting solution can be filtered again into a sodium bicarbonate solution. If desired, the resulting reaction solution can be dialyzed in water using a cellulose membrane and the composition can be lyophilized and isolated. The content of paclitaxel in the resulting composition may be determined by UV spectrometry.

In some embodiments, glucosamine, a group comprising glucosamine, a drug, a group comprising a drug, an agent (e.g., the agents described herein), and/or a group comprising an agent can be reacted with an amino acid such as glutamic and/or aspartic acid in which the glucosamine, a group comprising glucosamine, a drug, a group comprising a drug, an agent (e.g., the agents described herein), and/or a group comprising an agent is coupled (e.g., covalently bonded) to the amino acid. The resulting compound can then be reacted with polyglutamic acid or its salt to form one of the compositions described herein. In some embodiments, paclitaxel can be reacted with glutamic acid to form a compound in which the paclitaxel is covalently bonded to the pendant carboxylic acid group of the glutamic acid. The glutamic acid-paclitaxel compound can then be reacted with polyglutamic acid or its salt to form one of the compositions described herein. In some embodiments, paclitaxel can be reacted with aspartic acid to form a compound in which the paclitaxel is covalently bonded to the pendant carboxylic acid group of the aspartic acid. The aspartic acid-paclitaxel compound can then be reacted with polyglutamic acid or its salt to form the composition. If desired, the paclitaxel coupled to the amino acid by the C2′-oxygen can be separated from the paclitaxel coupled to the amino acid by the C7-oxygen using known separation methods (e.g., HPLC).

After formation of the composition, any free amount of agent (e.g., first drug) not covalently bonded to the polymer conjugate may also be measured. For example, thin layer chromatography (TLC) may be used to confirm the substantial absence of free paclitaxel remaining in the composition.

In some embodiments, a suitable polymeric reactant capable of forming a composition described herein can be operatively associated with a polydentate ligand. Suitable polydentate ligands include, but are not limited to, diethylenetriaminepentacetic acid (DTPA), tetraazacyclododecane-1,4,7,10-tetraacetic acid (DOTA), (1,2-ethanediyldinitrilo)tetraacetate (EDTA), ethylenediamine, 2,2′-bipyridine (bipy), 1,10-phenanthroline (phen), 1,2-bis(diphenylphosphino)ethane (DPPE), 2,4-pentanedione (acac), and ethanedioate (ox). Appropriate solvents, coupling agents, catalysts, and/or buffers as generally known to those skilled in the art and/or described herein may be used to form the composition. In other embodiments, a suitable polymeric reactant capable of forming a composition described herein can be operatively associated with a polydentate ligand precursor with protected oxygen atoms. As with polyglutamic acid, both the salt or acid form of the polymer obtained from polyglutamic acid and/or salt and an amino acid can be used as starting material for forming the composition.

In some embodiments, the polydentate ligand can be DTPA. In other embodiments, the polydentate ligand can be DOTA. In some embodiments, the polydentate ligand such as DTPA (with or without protected oxygen atoms), preferably in the presence of a coupling agent (e.g., DCC) and a catalyst (e.g., DMAP), can be reacted in a solvent (e.g, an aprotic solvent such as DMF). If protecting groups are present, removal can achieved using suitable methods. For example, the composition with the polydentate ligand precursor with protected oxygen atoms such as DTPA with oxygen atoms protected by t-butyl groups can be treated with acid such as trifluoroacetic acid. After removal of the protecting groups, the acid can be removed by rotary evaporation. In some embodiments, DTPA can be treated with a suitable base to remove the hydrogen atoms on the carboxylic acid —OH groups. In some embodiments, the base is sodium bicarbonate.

In some embodiments, a suitable polymeric reactant capable of forming a composition described herein can be operatively associated with a targeting agent. Exemplary targeting agents include, but are not limited to, arginine-glycine-aspartate (RGD) peptides, fibronectin, folate, galactose, apolipoprotein, insulin, transferrin, fibroblast growth factors (FGF), epidermal growth factors (EGF), and antibodies. Targeting agents can be chosen such that they interact with particular receptors. For example, a targeting agent can be chosen so that it interacts with one or more of the following receptors: αᵥ,β₃-integrin, folate, asialoglycoprotein, a low-density lipoprotein (LDL), an insulin receptor, a transferrin receptor, a fibroblast growth factor (FGF) receptor, an epidermal growth factor (EGF) receptor, and an antibody receptor. In some embodiments, the arginine-glycine-aspartate (RGD) peptide is cyclic (fKRGD).

Both the salt or acid form of the polymeric reactant capable of forming a composition described herein can be used as starting material for forming the composition with a targeting agent. In some embodiments, the targeting agent preferably in the presence of a coupling agent (e.g., DCC) and a catalyst (e.g., DMAP), can be reacted with the composition obtained from polyglutamic acid and/or salt and an amino acid in a solvent (e.g., an aprotic solvent such as DMF). After formation of the composition, any free amount of agent not covalently bonded to the composition may also be measured. For example, thin layer chromatography (TLC) may be used to confirm the substantial absence of any free targeting agent. Suitable methods known to those skilled in the art can be used to isolate and/or purify the composition (e.g., lypholization).

In some embodiments, a suitable polymeric reactant capable of forming a composition described herein can be operatively associated with a magnetic resonance imaging agent. In some embodiments, the magnetic resonance imaging agent can comprise a Gd(III) compound. One method for forming the magnetic resonance imaging agent is by reacting a paramagnetic metal with the polymer conjugate comprising a polydentate ligand. Suitable paramagnetic metals include but are not limited to Gd(III), Indium-III, and Yttrium-88. For example, a composition comprising DTPA can be treated with Gd(III) in a buffer solution for a period of several hours. Suitable methods known to those skilled in the art can be used to isolate and/or purify the composition. For instance, the resulting reaction solution can be dialyzed in water using a cellulose membrane and the composition can be lyophilized and isolated. The amount of paramagnetic metal may be quantified by inductively coupled plasma-optical emission spectroscopy (ICP-OES) measurement.

In some embodiments, a suitable polymeric reactant capable of forming a composition described herein can be operatively associated with a stabilizing agent. In some embodiments, the stabilizing agent can be polyethylene glycol. In one method, the stabilizing agent, preferably in the presence of a coupling agent (e.g., DCC) and a catalyst (e.g., DMAP), can be reacted with the composition obtained from polyglutamic acid and/or salt and an amino acid in a solvent (e.g., an aprotic solvent such as DMF). Progress of the reaction can be measured by any suitable method such as TLC. The resulting composition can be purified using methods known to those skilled in the art such as dialysis.

The compositions described herein may be formed into nanoparticles in aqueous solution. Such nanoparticles may be used to deliver a first drug to a selected tissue. In some embodiments, the composition is administered to the mammal by injection. In some embodiments, the composition is administered locally to the pancreas, lung, breast, colon, ovary, prostate, skin, kidney, liver, or spleen.

In some embodiments, the compositions describe herein further include and at least one selected from a pharmaceutically acceptable excipient, a carrier, and a diluent.

The term “diluent” refers to chemical compounds diluted in water that will dissolve a polymer conjugate described herein as well as stabilize the biologically active form of a polymer conjugate described. Salts dissolved in buffered solutions are utilized as diluents in the art. As used herein, an “excipient” refers to an inert substance that is added to a polymer conjugate described to provide, without limitation, bulk, consistency, stability, binding ability, lubrication, disintegrating ability, etc., to the composition. A “diluent” is a type of excipient.

The compositions described herein may have many different uses. In some embodiments, the compositions described herein may be used to deliver an imaging agent, targeting agent, magnetic resonance imaging agent, glucosamine, and/or a drug to a selected tissue. For example, compositions that include the Texas Red dye may be used to deliver an imaging agent to a selected tissue.

Some embodiments provide for a method of ameliorating the anti-clotting properties of a drug that can include operatively associating glucosamine with the drug. In some embodiments where the drug is attached to a polymer conjugate, the method can include operatively associating glucosamine with the polymer conjugate. In some embodiments, the methods for making the compositions described herein can be used to operatively associate the glucosamine with the polymer conjugate. In some embodiments, the drug can be a cancer drug, such as paclitaxel.

Embodiments described herein provide a method of treating or ameliorating a disease or condition comprising administering an effective amount of one or more compositions described herein or the pharmaceutical composition described herein to a mammal in need thereof. Other embodiments provide a use of an effective amount of one or more compositions described herein or the pharmaceutical composition described herein for treating or ameliorating a disease or condition. Yet other embodiments provide for a method of treating or ameliorating a disease or condition and/or treating or ameliorating the occurrence or risk of petechiae, comprising administering an effective amount of one or more compositions described herein or the pharmaceutical composition described herein to a mammal in need thereof. In some embodiments, the disease or condition can be a tumor, such as a lung tumor, breast tumor, colon tumor, ovarian tumor, prostate tumor, and melanoma tumor. In some embodiments, the disease or condition can be cancer, for example, lung cancer, breast cancer, colon cancer, ovarian cancer, prostate cancer, and melanoma.

Embodiments described herein provide a method of diagnosing a disease or condition comprising administering an effective amount of one or more compositions described herein or the pharmaceutical composition described herein to a mammal in need thereof. Other embodiments provide a use of an effective amount of one or more compositions described herein or the pharmaceutical composition described herein for diagnosing a disease or condition. In some embodiments, the disease or condition can be a tumor, such as a lung tumor, breast tumor, colon tumor, ovarian tumor, prostate tumor, and melanoma tumor. In some embodiments, the disease or condition can be cancer, for example, lung cancer, breast cancer, colon cancer, ovarian cancer, prostate cancer, and melanoma.

Some embodiments provide a method of imaging a portion of tissue comprising contacting a portion of tissue with an effective amount of one or more compositions described herein or the pharmaceutical composition described herein. Other embodiments provide a use of an effective amount of one or more compositions described herein or the pharmaceutical composition described herein for imaging a portion of tissue. In some embodiments, the tissue being imaged can be tissue from lung tumor, breast tumor, colon tumor, ovarian tumor, prostate tumor, and/or melanoma tumor.

In some embodiments, the mammal has been diagnosed as suffering from cancer, e.g., melanoma. In these embodiments, the compositions described herein can be administered to the mammal at a dose in the range of about 40 mg of first drug equivalents/kg (e.g., 40 mg of paclitaxel equivalents/kg) to about 345 mg of first drug equivalents/kg. In other embodiments, the compositions described herein can be administered to the mammal at a dose in the range of about 40 mg of first drug equivalents/kg (e.g., 40 mg of paclitaxel equivalents/kg) to about 550 mg of first drug equivalents/kg. In some embodiments, the person suffering from cancer may have been identified by expression profiling of cancer marker genes obtained from at least one tissue selected from pancreatic tissue, lung tissue, breast tissue, colon tissue, ovary tissue, prostate tissue, skin tissue, kidney tissue, liver tissue, and spleen tissue.

Techniques for formulation and administration of compositions that can include at least one selected from a pharmaceutically acceptable excipient, a carrier, and a diluent may be found in “Remington's Pharmaceutical Sciences,” Mack Publishing Co., Easton, Pa., 18th edition, 1990. The compositions may be manufactured in a manner that is itself known. Compositions may be formulated in any conventional manner using one or more physiologically acceptable pharmaceutical carriers comprising excipients and auxiliaries which facilitate processing of the active compounds into preparations which can be used pharmaceutically. Proper formulation is dependent upon the route of administration chosen. Any of the well-known techniques, pharmaceutical carriers, and excipients may be used as suitable and as understood in the art; e.g., in Remington's Pharmaceutical Sciences, above.

Multiple techniques of administering a composition exist in the art. Multiple techniques of administering a pharmaceutical composition exist in the art. Suitable routes of administration may include, for example, parenteral delivery, including intramuscular, subcutaneous, intravenous, intramedullary injections, as well as intrathecal, direct intraventricular, intraperitoneal, intranasal, or intraocular injections. The composition can also be administered in sustained or controlled release dosage forms, including depot injections, osmotic pumps, and the like, for prolonged and/or timed, pulsed administration at a predetermined rate. Additionally, the route of administration may be local or systemic.

Compositions suitable for administration (e.g., the composition that can include a polymer conjugate and glucosamine operatively associated with the polymer conjugate) include compositions where the active ingredients are contained in an amount effective to achieve its intended purpose. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, an effective amount means an amount of compound effective to prevent, alleviate or ameliorate symptoms of disease or prolong the survival of the subject being treated. Determination of an effective amount is well within the capability of those skilled in the art, especially in light of the detailed disclosure provided herein.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration.

Polymer conjugates disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. When selecting a model to determine efficacy, the skilled artisan can be guided by the state of the art to choose an appropriate model, dose, and route of administration, and regime.

### EXAMPLES

The following examples are provided for the purposes of further describing the embodiments described herein, and do not limit the scope of the invention.

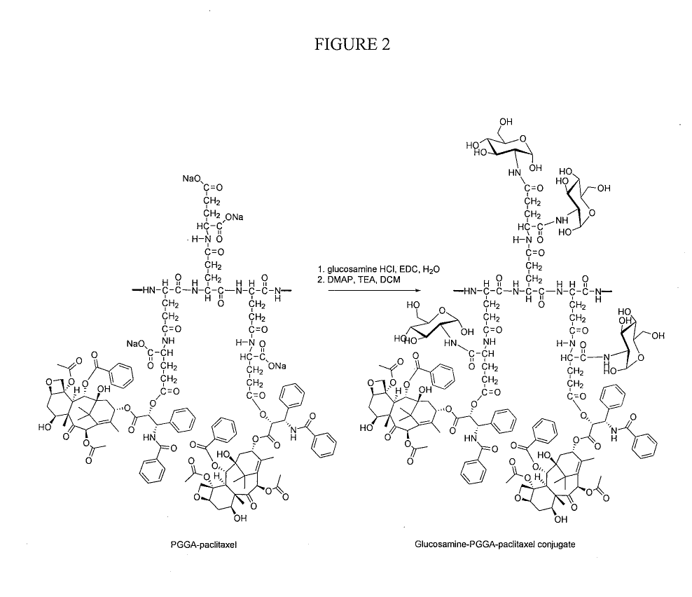

Polyglutamic acid (PGA) and other chemical reagents were purchased from Sigma-Aldrich chemical company. Paclitaxel (PTX) was purchased from NuBlock chemical company. Poly-(gamma-L-glutamylglutamine) (PGGA) and PGGA-paclitaxel conjugate having the structure illustrated in FIG. 2 were synthesized according to the procedures provided in U.S. Patent Publication No. 2007/0128118, which is hereby incorporated herein by reference in its entirety and particularly for the purpose of describing such materials and procedures. PGA-PTX was synthesized according to Auzenne et al., “Superior therapeutic profile of poly-L-Glutamic acid-paclitaxel copolymer Compared with Taxol in Xenogenic compartmental models of Human Ovarian Carcinoma,” Clinical Cancer Research 2002, 8, 573-581, for testing purposes only.

### Example 1

### Synthesis of Composition Including PGGA-Paclitaxel Conjugate and Saturated Glucosamine (Relative to PGGA)

PGGA-paclitaxel conjugate (2.0 g), glucosamine HCl (1.90 g), and 1-ethyl-3-(3-dimethylaminopropyl) carbodiimide (EDC) (2.2 g) were mixed in water (100 mL) in a 400-mL round bottom reaction flask equipped with a Teflon magnetic stir bar and while under an argon atmosphere. 4-Dimethylaminopyridine (DMAP) (0.14 g) and triethylamine (TEA) (1.2 mL) were dissolved in dichloromethane (DCM) (50 mL) and then added to the reaction flask. The reaction mixture was stirred for 24 hours. The reaction scheme is illustrated in FIG. 2. The aqueous phase was transferred to a separation funnel and washed with DCM (2×80 mL). The aqueous solution was poured into dialysis tubing (having a molecular weight cut-off of 10,000 Da) and first dialyzed against 0.05 M HCl (4 L) for one hour and then dialyzed against deionized (DI) water (4 L) for one hour. A 100% water change was performed and the solution was dialyzed for another hour. This process was repeated once more and allowed to dialyze overnight. The dialyzed solution was filtered through a Grade No. 50 Whatman filter and lyophilized to remove water. The resulting glucosamine-PGGA-paclitaxel composition was obtained at 40% yield and verified with ¹H-NMR spectroscopy.

### Example 2

### Synthesis of Composition Including PGGA-Paclitaxel and 10 Mol % Glucosamine (Relative to PGGA)

PGGA-PTX (100 mg) was partially dissolved in dimethylformamide (DMF) (5 mL) to form a solution. EDC (100 mg) and N-hydroxysuccinimide (NHS) (70 mg) were added into the solution to form a reaction mixture. The reaction mixture was stirred for 20 hours. A solution of glucosamine hydrochloric acid (5.3 mg) and triethylamine (TEA) (100 μL) in water (1 mL) was added to the reaction mixture and stirred for 20 hours. No free glucosamine was detected by a ninhydrin test. The reaction mixture was diluted with water (5 mL) and acidified with 1 N hydrochloric acid (2 mL). Precipitate formed and residue was collected by centrifugation and washed with water. The resulting 10% Glucosamine-PGGA-PTX was redissolved in a 0.2 M sodium bicarbonate solution and dialyzed against water (4 L). The water was changed 4 times. The product was lyophilized and obtained in 50% yield. The identity of the product was confirmed by ¹H-NMR spectroscopy.

### Example 3

### Synthesis of Composition Including PGGA-Paclitaxel and 25 Mol % Glucosamine (Relative to PGGA)

PGGA-PTX (100 mg) was partially dissolved in DMF (5 mL) to form a solution. EDC (100 mg) and NHS (70 mg) were added into the solution to form a reaction mixture. The reaction mixture was stirred for 20 hours. A solution of glucosamine hydrochloric acid (13.2 mg) and triethylamine (TEA) (100 μL) in water (1 mL) was added to the reaction mixture and stirred for 20 hours. No free glucosamine was detected by a ninhydrin test. The reaction mixture was diluted with water (5 mL) and acidified with 1 N hydrochloric acid (2 mL). Precipitate formed and residue was collected by centrifugation and washed with water. The resulting 25% Glucosamine-PGGA-PTX was redissolved in 0.2 M sodium bicarbonate solution and dialyzed against water (4 L). The water was changed 4 times. The product was lyophilized and obtained in 50% yield. The identity of the product was confirmed by ¹H-NMR spectroscopy.

### Example 4

### Synthesis of Composition Including PGGA-Paclitaxel and 50 Mol % Glucosamine (Relative to PGGA)

PGGA-PTX (100 mg) was partially dissolved in DMF (5 mL) to form a solution. EDC (100 mg) and NHS (70 mg) were added into the solution to form a reaction mixture. The reaction mixture was stirred for 20 hours. A solution of glucosamine hydrochloric acid (26.3 mg) and triethylamine (TEA) (100 μL) in water (1 mL) was added to the reaction mixture and stirred for 20 hours. No free glucosamine was detected by a ninhydrin test. The reaction mixture was diluted with water (5 mL) and acidified with 1 N hydrochloric acid (2 mL). Precipitate formed and residue was collected by centrifugation and washed with water. The resulting 50% Glucosamine-PGGA-PTX was redissolved in 0.2 M sodium bicarbonate solution and dialyzed against water (4 L). The water was changed 4 times. The product was lyophilized and obtained in 50% yield. The identity of the product was confirmed by ¹H-NMR spectroscopy.

### Example 5

### Synthesis of Composition Including PGGA-Paclitaxel and 75 Mol % Glucosamine (Relative to PGGA)

PGGA-PTX (100 mg) was partially dissolved in DMF (5 mL) to form a solution. EDC (100 mg) and NHS (70 mg) were added into the solution to form a reaction mixture. The reaction mixture was stirred for 20 hours. A solution of glucosamine hydrochloric acid (39.5 mg) and triethylamine (TEA) (200 μL) in water (1 mL) was added to the reaction mixture and stirred for 20 hours. No free glucosamine was detected by a ninhydrin test. The reaction mixture was diluted with water (5 mL) and acidified with 1 N hydrochloric acid (2 mL). Precipitate formed and residue was collected by centrifugation and washed with water. The resulting 75% Glucosamine-PGGA-PTX was redissolved in 0.2 M sodium bicarbonate solution and dialyzed against water (4 L). The water was changed 4 times. The product was lyophilized and obtained in 50% yield. The identity of the product was confirmed by ¹H-NMR spectroscopy.

### Example 6

### Activated Partial Thromboplastin (APTT) Test

The activated partial thromboplastin time (APTT) test is used as a general screening test for the detection of coagulation abnormalities in the intrinsic pathway. The composition prepared in Example 1 was dissolved in saline to a concentration of either 5 mg/mL or 10 mg/mL to form a solution. Other reagents included normal human plasma (George King, Biomedical Inc.), 0.025 M calcium chloride (Diagnostica Stago, Cat. # 104676), PTTA5 reagent (Diagnostica Stago, Cat. # 104859), and Coat Control N. (Diagnostica Stago, Cat. # 104695).

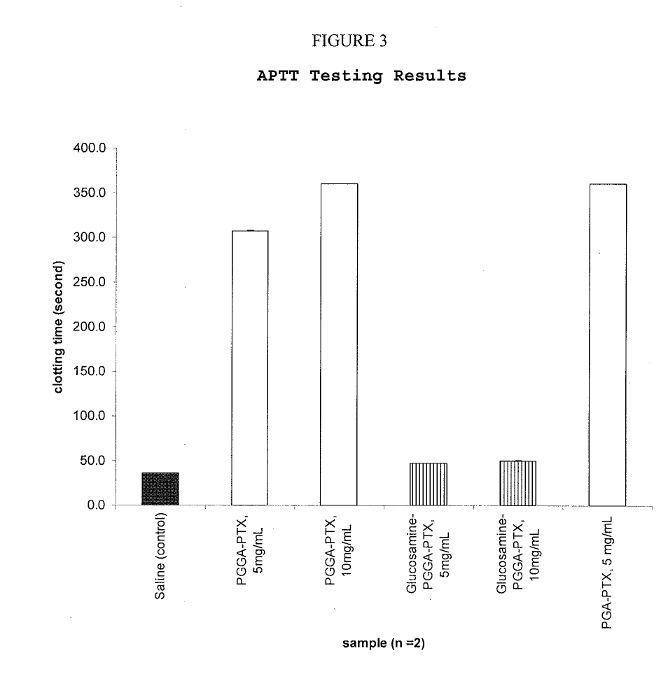

Normal human plasma (120 μL) and the testing composition solutions (30 μL) were added to a reaction cuvette. The PTT test was immediately performed using the STart® 4 Coagulation Analyzer (Diagnostica Stago), pursuant to the manufacturer's instructions. 0.9% NaCl (30 μL) was used as a control in place of the testing composition solutions. The results were shown in FIG. 3. As shown in FIG. 3, the clotting time for a composition that includes glucosamine, PGGA, and PTX is about 50-60 seconds. In contrast, otherwise comparable compositions that lack glucosamine exhibit a clotting time of over 300 seconds.

The APTT for an untreated sample of human plasma is about 40 seconds, whereas the APTT of human plasma treated with an anionic PGGA-paclitaxel polymer conjugate is about 350 seconds. As described herein, it has been advantageously and unexpectedly discovered that operative association of glucosamine to the anionic PGGA-paclitaxel polymer conjugate can significantly reduce the coagulation time of the drug treated human plasma, e.g., from about 350 seconds to about 50-60 seconds. Accordingly, polymer conjugate compositions described herein may exhibit less of an anticoagulant effect and/or may exhibit a reduced occurrence of petechiae as compared to control compositions that lack glucosamine.

It will be understood by those of skill in the art that numerous and various modifications can be made without departing from the spirit of the present invention. Therefore, it should be clearly understood that the forms of the present invention are illustrative only and not intended to limit the scope of the present invention.

What is claimed is:

1. A composition comprising:
a polymer conjugate comprising at least one recurring unit selected from the group consisting of Formula (I) and Formula (II): 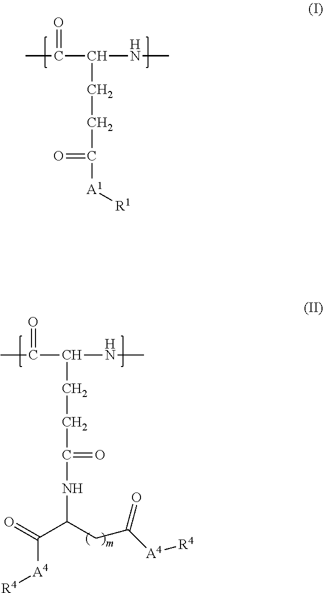
A¹ and A⁴ are each independently oxygen or NR⁷, wherein R⁷ is hydrogen or C₁₋₄ alkyl;
R¹ is a group that comprises a first drug;
each R⁴ is independently hydrogen, a group that comprises a first drug, a group that comprises glucosamine, ammonium, or an alkali metal, with the proviso that at least one of the R⁴ is a group that comprises a first drug and at least one of the R⁴ is hydrogen, a group that comprises a first drug, a group that comprises glucosamine, ammonium, or an alkali metal;
each m is 1 or 2;
wherein the polymer conjugate is operatively associated with glucosamine.

2. The composition of claim 1, wherein the polymer conjugate further comprises at least one recurring unit selected from the group consisting of Formula (III) and Formula (IV): 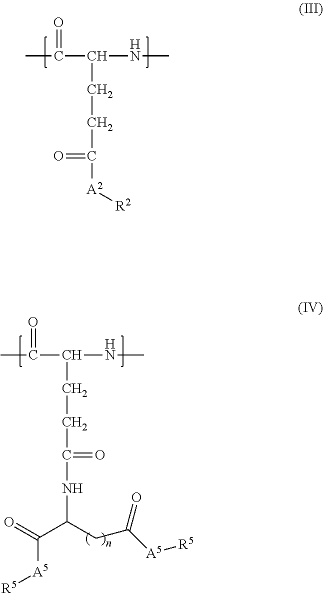
wherein:
A² and A⁵ are each independently oxygen or NR⁷, wherein R⁷ is hydrogen or C₁₋₄ alkyl;
R² is a group that comprises glucosamine;
each R⁵ is independently hydrogen, a group that comprises glucosamine, ammonium, or an alkali metal, with the proviso that at least one R⁵ is a group that comprises glucosamine; and
n is 1 or 2.

3. The composition of claim 2, wherein the composition comprises at least one recurring unit of Formula (I) and at least one recurring unit of Formula (III).

4. The composition of claim 2, wherein the composition comprises at least one recurring unit of Formula (II) and at least one recurring unit of Formula (IV).

5. The composition of claim 1, wherein the first drug is an anticancer drug.

6. The composition of claim 5, wherein the anticancer drug is selected from the group consisting of paclitaxel, docetaxel, camptothecin and doxorubicin.

7. The composition of claim 1, wherein the composition comprises an amount of glucosamine in the range of from about 1 mole % to about 90 mole %.

8. The composition of claim 1, wherein the composition comprises an amount of glucosamine in the range of from about 50 mole % to about 80 mole %.

9. The composition of claim 1, wherein the composition comprises an amount of glucosamine in the range of from about 10 mole % to about 70 mole %.

10. The composition of claim 1, wherein the composition comprises an amount of glucosamine that is effective to yield a composition that exhibits a reduced APTT as compared to an otherwise comparable composition that lacks glucosamine.

11. The composition of claim 1, wherein the polymer conjugate further comprises at least one recurring unit selected from the group consisting of Formula (V) and Formula (VI): 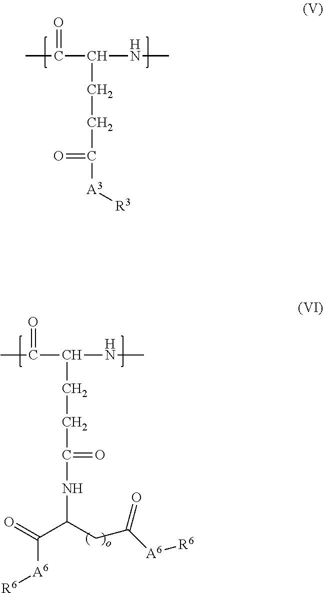
wherein:
A³ and A⁶ are each independently oxygen or NR⁷, wherein R⁷ is hydrogen or C₁₋₄ alkyl;
R³ and R⁶ are each independently selected from the group consisting of a hydrogen, a C₁₋₁₀ alkyl group, a C₆₋₂₀ aryl group, an ammonium group, an alkali metal, a polydentate ligand, a polydentate ligand precursor with protected oxygen atoms, a group that comprises a targeting agent, a group that comprises an optical imaging agent, a group that comprises a magnetic resonance imaging agent, and a group that comprises a stabilizing agent; and
o is 1 or 2.

12. The composition of claim 11, comprising at least one recurring unit of Formula (I), at least one recurring unit of Formula (III), and at least one recurring unit of Formula (V).

13. The composition of claim 11, comprising at least one recurring unit of Formula (II), at least one recurring unit of Formula (IV), and at least one recurring unit of Formula (VI).

14. The composition of claim 11, wherein the alkali metal is sodium.

15. A method of making the composition of claim 1, comprising:
dissolving or partially dissolving a polymeric reactant comprising at least one recurring unit selected from the group consisting of Formula (VII) and Formula (VIII) in a solvent to form a dissolved or partially dissolved polymeric reactant; 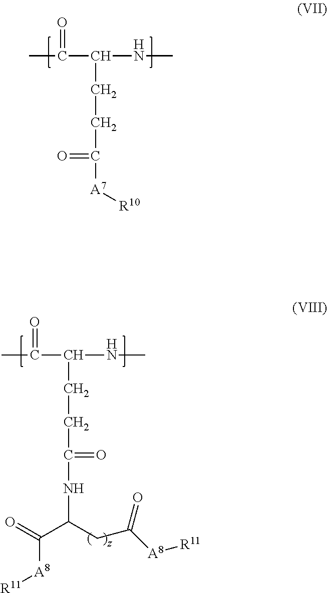
wherein:
z is independently 1 or 2;
A⁷ and each A⁸ are oxygen; and
R¹⁰ and each R¹¹ are independently selected from the group consisting of hydrogen, ammonium, and an alkali metal;
reacting the dissolved or partially dissolved polymeric reactant with a second reactant, wherein the second reactant comprises the first drug; and
intermixing the dissolved or partially dissolved polymeric reactant with a third reactant, wherein the third reactant comprises glucosamine.

16. The method of claim 15, wherein the first drug is an anticancer drug selected from the group consisting of a taxane, camptothecin, and doxorubicin.

17. The method of claim 15, wherein the first drug is a taxane selected from the group consisting of paclitaxel and docetaxel.

18. A method of making the composition of claim 11, comprising:
dissolving or partially dissolving a polymeric reactant comprising at least one recurring unit selected from the group consisting of Formula (VII) and Formula (VIII) in a solvent to form a dissolved or partially dissolved polymeric reactant; 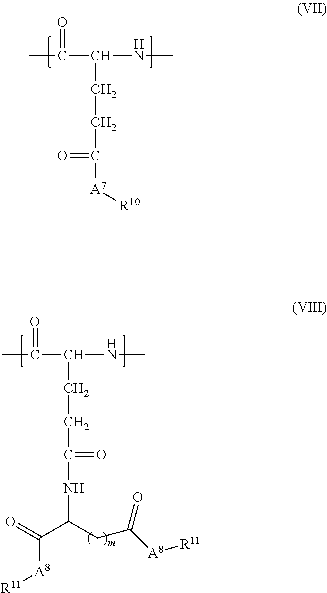
wherein:
z is independently 1 or 2;
A⁷ and each A⁸ are oxygen; and
R¹⁰ and each R¹¹ are independently selected from the group consisting of hydrogen, ammonium, and an alkali metal;
reacting the dissolved or partially dissolved polymeric reactant with a second reactant, wherein the second reactant comprises the first drug;
intermixing the dissolved or partially dissolved polymeric reactant with a third reactant, wherein the third reactant comprises glucosamine; and
reacting the dissolved or partially dissolved polymeric reactant with a fourth reactant, wherein the fourth reactant comprises at least one selected from the group consisting of a polydentate ligand, a polydentate ligand precursor with protected oxygen atoms, a group that comprises a targeting agent, a group that comprises an optical imaging agent, a group that comprises a magnetic resonance imaging agent, and a group that comprises a stabilizing agent.

19. A method of treating, ameliorating, or diagnosing a disease or condition comprising administering an effective amount of the composition of claim 1 to a mammal in need thereof.

20. The method of claim 19, wherein the disease or condition is selected from the group consisting of pancreatic cancer, lung cancer, melanoma, kidney cancer, liver cancer and spleen cancer.

21. The method of claim 19, wherein the composition is administered to the mammal by injection.
